(19) United States
(12) Patent Application Publication
(10) Pub. No.: US 2003/0036761 A1
(43) Pub. Date: Feb. 20, 2003

(54) Pharmacolgical sleeve

(76) Inventors: Crista Smothers, Cordova, TN (US); John Schneider, Memphis, TN (US); Ed Austin, Bartlett, TN (US); Ajit Mishra, San Antonio, TX (US); Mark Gosney, Memphis, TN (US); Anthony James, Bartlett, TN (US); David Castleman, Bartlett, TN (US); Mark Hamby, Bartlett, TN (US); Julie Bearcroft, Bartlett, TN (US); Jeff Holbrook, Memphis, TN (US); Jeff Schryver, Memphis, TN (US); Michael Cooper, Memphis, TN (US)

Correspondence Address: CHIEF PATENT COUNSEL, SMITH & NEPHEW, INC., 1450 BROOKS ROAD, MEMPHIS, TN, 38116, US

(21) Appl. No.: 10/189,862

(22) Filed: Jul. 3, 2002

Related U.S. Application Data
(60) Provisional application No. 60/303,715, filed on Jul. 9, 2001.

(51) Int. Cl. A61B 17/58

(52) U.S. Cl. 606/72

(57) ABSTRACT

This invention relates to methods, devices, and systems for preventing complications, including for example, infection associated with any percutaneous or external fixation of bones by placing a pharmacological sleeve over an external fixation element. The pharmacological sleeve may be comprised of a polymer sleeve having one or more internal splines along at least part of the inner surface of the sleeve. The sleeve may be suffused or coated on any or all surfaces with a pharmacological coating which allows accumulation of drugs in the tissue surrounding the external fixation element, thereby reducing potential complications.

### RELATED APPLICATION DATA

This document claims the benefit of U.S. Provisional Application No. 60/303,715, filed Jul. 9, 2001.

### FIELD OF THE INVENTION

This invention generally relates to methods, systems, and devices for preventing complications, such as infection, during the external fixation of bones and more particularly to a pharmacological sleeve for use with external fixation elements to help prevent complications during the external fixation of bones.

### BACKGROUND

External fixation systems are used in the treatment of traumatic injury, post traumatic injury, and congenital deformity correction. These systems orient and immobilize sections of a fractured bone during a period of osteogenesis and healing. Generally, external fixation elements protrude through the skin and underlying tissue to immobilize damaged bones and to facilitate the natural healing process of a bone in the proper anatomical position. Fixation elements may extend completely through the bone, exiting the skin on the opposite side of the bone, or may extend through the bony skeleton and out of only one side of the limb.

The term “external fixation element or member” as used herein includes external fixator pins, external fixator wires, and any other orthopedic pin or wire that breaks the skin surface and that may be used with an external fixation system, a cantilever structure, a tension structure or any other percutaneous bone stabilizer or fixator apparatus. Typically, external fixation elements are made of metal, such as stainless steel or titanium. External fixation elements may be circular, hexagonal, or any other multisided shape.

External fixator pins are commercially available in diameters ranging between 4 millimeters and 6.5 millimeters and in lengths ranging between 51 millimeters and 203 millimeters. The distal end of a pin typically has a self-drilling, self-tapping thread that is used to secure the pin to the bone. The proximal end of the pin is attached to the external fixation frame.

External fixator wires have smaller diameters than external fixator pins, typically between 1.5 millimeters and 2.8 millimeters. A wire is often provided with self-drilling flutes at the distal end, and is drilled completely through the bone, exiting through the skin on the opposite side of the bone. The bone wire is clamped to the external frame at one end, and then placed under tension before being clamped at the other end to another part of the frame. External frames suitable for use with bone wires often contain arcuate members surrounding or partially surrounding the bone for clamping the wire on both sides.

The process of attaching an external fixation element to a bone includes first making an incision in the skin surface and in the subcutaneous fat and muscle tissues surrounding the bone. The bone is then tapped to receive the threaded end of the external fixation element. The external fixation element is attached to the bone by inserting the threaded end of the external fixation element into the incision and screwing it into the tapped cavity located within the bone. Alternatively, the external fixation element may be self-tapping, whereby the external fixation element is attached to the bone by first drilling a cavity in the bone and then screwing an external fixation element into the cavity. Still yet, the external fixation element may be self-drilling, whereby the external fixation element is attached to the bone by simply screwing it into the bone.

At least two external fixation elements are installed in the bone at various locations in order to properly position the bone. Once the external fixation elements have been positioned within the bone, an adjustable alignment device is attached to each external fixation element and positioned so that the bone is in proper anatomical position for healing. The adjustable alignment device may take on many forms; however, regardless of its form, each device is capable of securing each external fixation element in a fixed position relative to each other throughout its tenure in the patient. The external fixation elements remain attached to the bone until the bone has healed. It is not uncommon for the external fixation elements to remain in a patient for more than two or three months.

During external fixation, the surrounding skin tissue does not attach to the external fixation elements because skin tissue, and other biological tissues, do not adhere to metals. Thus, an open wound surrounds the external fixation elements throughout their tenure in the bone. A common complication resulting from this open wound is an infection that occurs at the skin surface surrounding the open wound. Infection is the number one complication associated with external fixation systems. The incidence of infection associated with external fixation has been reported in the literature to range between 10 and 30%. An open wound surrounding a foreign body, such as an external fixation element, provides access for bacteria and an increased risk of infection at the wound site. Exudate often seeps from the external fixation element insertion site onto the skin. The exudate picks up skin flora that can diffuse back into the wound along the external fixation element surface causing infection. The potential for infection is increased by any motion of the external fixation element into and out of the wound and by bacterial proliferation at the wound site. Moreover, the tissues contacting the external fixation element are in a state of chronic inflammation, impairing the normal immune response against bacterial proliferation.

Infections associated with external fixation may range from minor infections at the skin surface to deep infections of the bone, such as osteomyelitis. There have been some attempts to treat these infections by filling osteomyelitic cavities with polymer particles impregnated with an antibiotic. Another mode of treating infection is administering oral antibiotics. However, bacteria often adhere to the metal surfaces of external fixation elements and form a bacterial biofilm. Bacterial biofilm is a layer of bacteria contained in a slimy layer which protects bacteria from oral antibiotics and allows bacteria to reproduce freely.

Consequently, many patients who have infections must undergo hospitalization for administration of intravenous antibiotics, which occurs over a five-day period and is quite expensive. For severe infections in which oral and intravenous antibiotics have been ineffective, the external fixation elements have been removed to combat the infection. Such severe infections occur in as many as 6-12% of patients. The premature removal of the external fixation elements can have adverse effects on the healing process.

Prophylaxis for infection includes applying ointments to the wound, pouring saline over the wound, and covering the wound with antiseptic lotions. These treatments often cross-contaminate wounds, seal in infectious organisms, irritate the skin surrounding the wound, and require numerous clinic visits. There is a continuing need for improved methods of preventing infection associated with external fixation.

Occlusive, moist, liquid-absorbent, or antimicrobial wound dressings have been used to prevent infection. Some have attempted to prevent infection by draining wound secretions. An antibacterial hose with a slit along its entire length has been used to drain wound secretions. Similarly, an antibiotic-containing cylinder with a central longitudinal duct and a plurality of radial outlet openings along its entire length can be slid over external fixation elements to drain wound secretions. The cylinder is introduced into wound sites so that generated wound secretions can be diverted from the wound site through the radial outlet openings. The cylinder has a diameter of approximately 5.6 millimeters, and it rigidly surrounds the external fixation element.

Although the hose and the cylinder allow improved drainage of wound secretions, neither the hose nor the cylinder effectively prohibits bacterial access to the open wound surrounding the external fixation element. In fact, the cylinder, with its smooth inner walls and rigid fit may assist the adhesion of bacteria to the metal surface of the external fixation element by protecting the bacteria from the body's immune system and from oral or parenteral antibiotic therapy. Further, the smooth inner walls of the cylinder do not allow for maximum elution of the antimicrobial agent. Therefore, there remains a need for a device that prevents bacterial adhesion to the external fixation element and provides maximum accumulation of an antimicrobial agent at the wound site.

Coating or infusing medical devices with antimicrobial agents, which allows prolonged release of antibiotics into the surrounding wound tissue, has been used to prevent infection. For example, a catheter may have a silicone outer sheath and a pharmacologically active antimicrobial agent sandwiched by an inner layer of silicone that forms the lumen of the catheter. The pharmacologically active agent may have antimicrobial properties, and the outer and inner silicone layers are permeable to the pharmacologically active agent, allowing diffusion of the active ingredient into the lumen of the device and to the exterior surface of the device. Unlike indwelling catheters, external fixation elements are not hollow and do not have lumens. Further, external fixation elements are not permeable to pharmacological agents. Therefore, there remains a need to provide pharmacological agents to tissues surrounding external fixation element sites.

Other efforts at preventing infection have focused on applying antimicrobial devices around an external fixation element. An antimicrobial device surrounds an external fixation element at a point where the external fixation element enters a patient's skin surface and preferably extends along the external fixation element on either side of the skin surface. The antimicrobial device further includes a cuff that is attached around the outside surface of the device and preferably positioned slightly beneath the skin surface once the antimicrobial device and the external fixation element have been installed in a patient. Allegedly, the cuff promotes growth of scar tissue into the interstices of the cuff. Further, the device is preferably composed of medical tubing and is kept in position using an interference fit, whereby the internal diameter of the antimicrobial device is smaller than the external diameter of the external fixation element to which the antimicrobial device is attached.

While an interference fit is a viable solution in other mechanical applications, such is not always the case here. An interference fit works well in situations where each part is produced using tight tolerances and accurate manufacturing methods and machines. However, such is not the case during the manufacturing of medical tubing. Instead, medical tubing is not manufactured to tight tolerances. Thus, when the medical tubing is produced nearer the smaller end of its broad tolerance range, a much greater amount of force must be used to position the antimicrobial device on the external fixation element. It is not uncommon for a person of ordinary strength to have difficulty positioning an antimicrobial device, having an interference fit, on an external fixation element due to the device having an inside diameter that is too small. Further, at times, it is impossible to position an antimicrobial device, having an interference fit, on an external fixation element.

An interference fit also does not accommodate the swelling that is part of the healing process, which will allow movement of the external fixation element and possible extrusion of the device by the swollen wound site. The tight interference fit also does not allow exudates and wound fluids to drain freely.

Further, an interference fit does not allow the accumulation of antibiotic between the device and the external fixation element. Consequently, bacteria are more likely to adhere to the metal surface of the external fixation element leading to the formation of bacterial biofilm on the external fixation element surface.

Devices having an interference fit are also more expensive to manufacture because they must be made in a wide range of sizes to accommodate all diameters of currently marketed external fixation elements.

Accordingly, there is a continuing need for improved prophylaxis of infection associated with external fixation elements. There is also a need to prevent other complications associated with bone fixation. There is also a need for pharmacological sleeves that provide a space between the sleeve and the external fixation element, which will allow improved wound drainage, accumulation of larger amounts of pharmacological agents at the wound site, and improved prevention of bacterial biofilm on the external fixation elements. There is also a need for a device that accommodates a wide range of diameters of external fixation elements but that is not expensive to manufacture.

### SUMMARY

The present invention is directed to methods, devices, and systems for preventing complications, such as infection, associated with the external fixation of bones by using a pharmacological sleeve. Pharmacological sleeves, according to some embodiment of this invention, are comprised of a polymer sleeve suffused or coated on at least some surfaces with a pharmacological coating. The sleeve is adapted to surround a percutaneous member that is used to control the position of fractured or malformed bone. In a preferred embodiment, the pharmacological sleeve has at least one internal spline or spacer on the inner surface of the sleeve, in order to provide space between the member and the sleeve. In another embodiment, the spline or spacer is a separate component from the sleeve. The pharmacological sleeve may be used with external fixator pins, external fixator wires, and any other orthopedic pin or wire that breaks the skin surface and that may be used with an external fixation system, a cantilever structure, a tension structure or any other percutaneous bone stabilizer or fixator apparatus.

Methods according to this invention involve using a gauge/pusher to determine the proper length of the sleeve. A grooved ruler may be used to measure the length of the sleeve, as determined using a gauge/pusher. A sleeve of a proper length may be placed over an external fixation element. Then, using a gauge/pusher, the sleeve may be manually pushed through the subcutaneous tissue to the implant point in the bone. The position of the sleeve on the external fixation element may be adjusted using a notched pusher. The pharmacological sleeve may be, but need not be, positioned on the external fixation element with a positioning mechanism.

An object of certain aspects of this invention is to prevent complications associated with the external fixation of bones.

An object of certain aspects of this invention is to provide improved prophylaxis for infections associated with the external fixation of bones.

Another object of certain aspects of this invention is to decrease the formation of bacterial biofilm on the surfaces of implanted external fixation elements.

Another object of certain aspects of this invention is to maximize drug accumulation at the wound site by providing prolonged release of pharmacologic agents at the wound site.

Another object of certain aspects of this invention is to maximize drug accumulation at the wound site by providing a channel for introducing pharmacologic agents at the wound site.

Another object of certain aspects of this invention is to provide improved wound drainage at the wound site.

Another object of certain aspects of this invention is to provide improved infection prophylaxis while accommodating swelling at the wound site.

Another object of certain aspects of this invention is to minimize clinic and hospital visits associated with infection prophylaxis.

Another object of certain aspects of this invention is to decrease the expenses associated with infection prophylaxis.

These and other objects, features, aspects, and advantages of this invention will be readily understood by those skilled in the art by reference to the remainder of this document.

### BRIEF DESCRIPTION OF THE DRAWINGS

The accompanying drawings, which are incorporated in and form a part of the specification, illustrate preferred embodiments of the present invention and, together with the description, disclose the principles and aspects of the invention.

FIG. 1 depicts an embodiment of a pharmacological sleeve placed on an external fixation element which is attached to a bone fixator apparatus.

FIG. 2 depicts a sectional view of an embodiment of a pharmacolgical sleeve placed over an external fixation element, both of which are inserted in to an implant point in bone.

FIG. 3 is a cross-sectional view of one embodiment of a pharmacolgical sleeve having a cylindrical shaped sleeve and three internal splines of varying shapes spaced 120° apart.

FIG. 4 is a cross-sectional view of one embodiment of a pharmacologial sleeve having a cylindrical shaped sleeve and three semicircular shaped internal splines spaced 120° apart and positioned on circular external fixation element.

FIG. 5 is a cross-sectional view of a pharmacological sleeve having an interference fit over an external fixation element.

FIG. 6 is a cross-sectional view of a pharmacological sleeve that fits loosely around an external fixation element.

FIG. 7 is a cross-sectional view of a heat shrink tapered pharmacological sleeve positioned on an external fixation element.

FIG. 8 is a cross-sectional view of a pharmacological sleeve, having a rolled edge attached on an external fixation element.

FIG. 9 is cross-sectional view of a pharmacological sleeve, having a coating on an end of the sleeve attached on an external fixation element.

FIG. 10 is a cross-sectional view of a pharmacological sleeve, having a crimped end attached on an external fixation element.

FIG. 11 is a cross-sectional view of a pharmacological sleeve, having a crimped end attached on an external fixation element.

FIG. 12 is a side plan view of a pharmacolgical sleeve, having a pigtail end, attached on an external fixation element.

FIG. 13 is a cross-sectional view of a pharmacological sleeve, having a heat shrinkable nose attached on an external fixation element.

FIG. 14 is a cross-sectional view of a wedge attached on an external fixation element having a pharmacological sleeve.

FIG. 15 is a cross-sectional view of a retainer ring attached on an external fixation element.

FIG. 16 is a cross-sectional view of a pharmacological sleeve, having heat shrink tubing attached over the sleeve, positioned on an external fixation element.

FIG. 17 is a front plan view of a hose clamp assembly attached on a pharmacological sleeve and an external fixation element.

FIG. 18 is a cross-sectional view of a tapered connector attached on a pharmacological sleeve and an external fixation element.

FIG. 19 is a cross-sectional view of a cap attached on a pharmacolgical sleeve and an external fixation element.

FIG. 20 is a cross-sectional view of an alternative embodiment of the cap shown in FIG. 19 which is attached to a pharmacological sleeve and an external fixation element.

FIG. 21 is a cross-sectional view of a retainer band attached on a pharmacological sleeve and an external fixation element.

FIG. 22 is a cross-sectional view of a toothed hinge clamp attached on a pharmacological sleeve and an external fixation element.

FIG. 23 is a cross-sectional view of a cap, containing an O-ring, attached on a pharmacological sleeve and an external fixation element.

FIG. 24 is a front plan view of a clip for sealing and positioning a pharmacological sleeve on an external fixation element.

FIG. 25A is a top plan view of a snap-on clip for sealing and positioning a pharmacological sleeve on an external fixation element.

FIG. 25B is a top plan view of an alternative embodiment of a snap-on clip for sealing and positioning a pharmacological sleeve on an external fixation element.

FIG. 26 is a perspective view of an O-ring attached on a pharmacological sleeve and an external fixation element.

FIG. 27 is a perspective view of a clip with a V-shaped body attached on a pharmacological sleeve and an external fixation element.

FIG. 28A is a top plan view of a C-clamp for sealing and positioning a pharmacological sleeve on an external fixation element.

FIG. 28B is a top plan view of an alternative embodiment of a C-clamp for sealing and positioning a pharmacological sleeve on an external fixation element.

FIG. 29 is a top plan view of a disk clip for sealing and positioning a pharmacological sleeve on an external fixation element.

FIG. 30 is a top plan view of a set screw clamp for sealing and positioning a pharmacological sleeve on an external fixation element.

FIG. 31 is a top plan view of a toothed clamp for sealing and positioning a pharmacolgical sleeve on an external fixation element.

FIG. 32 is a top plan view of an alternative embodiment of a toothed clamp for sealing and positioning a pharmacological sleeve on an external fixation element.

FIG. 33 is a top plan view of a chip clip for sealing and positioning a pharmacolgical sleeve on an external fixation element.

FIG. 34 is a perspective view of a 1.8 mm gauge/pusher used in one method according to this invention.

FIG. 35 is a perspective view of a 5 mm gauge/pusher used in one method according to this invention.

FIG. 36 is a perspective view of a 6 mm gauge/pusher used in one method according to this invention.

FIG. 37 depicts the insertion of a 1.8 mm gauge/pusher to release tissue from around the external fixation element according to one embodiment of this invention.

FIG. 38 depicts the use of a 1.8 mm gauge/pusher to measure the length of a pharmacological sleeve as the distance from the bone to the bone fixator apparatus according to one embodiment of this invention.

FIG. 39 depicts the use of a 5 or 6 mm gauge/pusher to measure the length of a pharmacological sleeve as the distance between the bone and the bone fixator apparatus according to one embodiment of this invention.

FIG. 40 is a front plan view of a grooved ruler and scissor used in one method according to this invention.

FIG. 41 depicts the placement of a pharmacological sleeve in the groove of a grooved ruler so that it is aligned with the measurement taken with the gauge/pusher according to one embodiment of this invention.

FIG. 42 depicts the placement of a pharmacological sleeve in the groove of a grooved ruler so that it is aligned with the measurement taken with the gauge/pusher according to one embodiment of this invention.

FIG. 43 depicts the placement of a pharmacological sleeve onto the end of an external fixation element according to one embodiment of this invention.

FIG. 44 depicts the use of the gauge/pusher to slide a pharmacological sleeve down the external fixation element to the implant point in the bone according to one embodiment of this invention.

FIG. 45 depicts the placement of a pharmacological sleeve onto an external fixation element and use of a gauge/pusher to push the sleeve down the fixation element until it is seated against the bone, according to one embodiment of this invention.

FIG. 46 is a front plan view of a notched pusher used in a method according to one embodiment of this invention.

FIG. 47 depicts the use of a notched pusher to maneuver a pharmacological sleeve back and forth on an external fixation element for positional adjustment according to one embodiment of this invention.

FIG. 48 depicts a pharmacological sleeve positioned on an external fixation element that has been tensioned by tightening nuts that are attached to a bone fixator apparatus according to one embodiment of this invention.

FIG. 49 depicts a pharmacological sleeve positioned on an external fixation element that has been tensioned by tightening nuts that are attached to a bone fixator apparatus according to one embodiment of this invention.

FIG. 50 depicts a pharmacological sleeve positioned on a 5 mm external fixation element and held in place by a clip with two interlocking arms according to one embodiment of this invention.

FIG. 51 depicts an embodiment of a pharmacological sleeve positioned on an external fixation element that is attached to a pulling-force according to one embodiment of this invention.

### DETAILED DESCRIPTION

Methods, systems, and devices according to this invention seek to provide a pharmacological sleeve for use with external fixation elements to aid in prevention of complications, such as infection, during the external fixation of bones. A pharmacological sleeve 110 having features of the present invention comprises a polymer sleeve 110 with at least one internal spline or spacer 114 on the inner surface 116 of the sleeve 110. The spline or spacer 114 may extend along a portion of the length or the entire length of the sleeve 110. The spacer 114 may be formed as part of the sleeve 110, such as extruded, or it may be a separate component. The spacer or spline 114 may be a straight longitudinal component or a helical structure. The spacer or spline 114 may be formed of a plurality of ridges or any other structures that provide space between the sleeve 110 and the member. The sleeve 110 may be suffused or coated on all surfaces with a pharmacological coating containing a drug-surfactant complex, such as gentamicin lauryl sulfate.

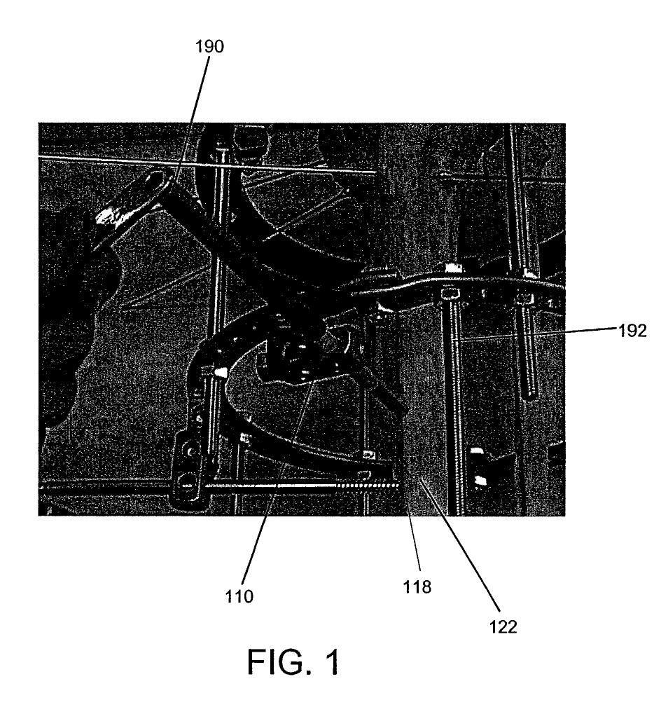

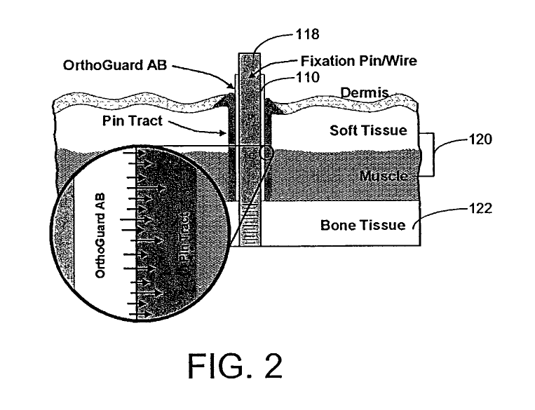

As shown in FIGS. 1 and 2, a preferred embodiment of a pharmacological sleeve 110 is placed over the external fixation element 118, manually pushed through the subcutaneous tissue 120, to the implant point in the bone 122, and the sleeve 110 may be secured with a positioning mechanism 124. The internal splines 114 of this sleeve 110 create and maintain a space 126 between the inner surface 116 of the sleeve 110 and the outer diameter of the external fixation element 118, which allows for improved wound drainage. The pharmacological coating allows prolonged release of the drugs into surrounding tissue 120 and into the space 126 created by the internal splines 114. The accumulation of pharmacological agents in this space 126 allows for improved infection prophylaxis and prevents the formation of bacteria biofilm on the external fixation elements 118.

A pharmacological sleeve 110 according to this invention includes a sleeve 110 having an inner surface 116 and an outer surface 128. The inner surface 116 and the outer surface 128 may be of any texture. In a preferred embodiment, the inner surface 116 and outer surface 128 are smooth surfaces, which aids in preventing bacterial growth sites on the sleeve 110 and allows easier removal of the sleeve 110. In one embodiment, the sleeve 110 comprises a polymer material, which may include, but is not limited to, thermoplastic polymers such as Tecoflex polyurethane. In one embodiment of the invention, the sleeve 110 is a hollow cylinder. In alternative embodiments, the sleeve 110 may be a square, rectangle, octagon, hexagon, oval or any other shape.

In one embodiment of a pharmacological sleeve 110 according to this invention, the length of the sleeve 110 is the maximum length for the longest external fixation element 118 that is available on the market. For example, external fixation elements 118 are available in lengths ranging from 51 to 203 millimeters. Therefore, the sleeve 110 may be 203 millimeters in length, and the sleeve 110 may be cut with scissors to desired lengths for use with shorter external fixation elements 118. The pharmacological sleeve 110 may be cut shorter than the length of the external fixation element 118 to allow a portion of the external fixation element 118 to be screwed into the bone 122 and to allow a positioning mechanism 124, further described below, to secure the external fixation element 118. The pharmacological sleeve 110 may be in any length shorter than the length of the external fixation element 118, limited only by the criteria that the pharmacological sleeve 110 be of sufficient length to span the opening in the human skin and extend into a patient's body a minimal distance and in the opposite direction a minimal distance to effectively ward off infection, as illustrated by FIG. 2. A pharmacological sleeve 110, according to one embodiment of this invention, is designed for use in external fixation for the treatment of animals.

The sleeve 110 can be provided in various diameters to match the diameters of currently marketed external fixation elements 118, such that the inner diameter of the sleeve 110 is larger than the outer diameter of the external fixation element 118. For example, external fixation elements 118 are available in diameters ranging from 1.5 to 6.5 millimeters. Therefore, the sleeve 110 is provided in diameters to accommodate external fixation elements 118 with diameters of 1.5 to 6.5 millimeters.

In one embodiment, an antimicrobial effect of the sleeve 110 is achieved with coating technology. In one embodiment of the invention, some or all surfaces of the sleeve 110 are coated with an antimicrobial coating. Antimicrobial coating that is suitable for use in the present invention is described in U.S. Pat. No. 5,525,348 to Whitbourne et al., which is a continuation of U.S. Pat. No. 5,069,899 to Whitbourne et al., both of which are incorporated herein by this reference. In another embodiment, any other suitable antimicrobial coating is used.

In one embodiment, a pharmacological coating applied to the sleeve 110 consists of a drug-surfactant ionic complex, nitrocellulose, and a polyurethane resin. In one embodiment, the drug-surfactant ionic complex is gentamicin lauryl sulfate. The drug-surfactant ionic complex may be pre-formed by dissolving gentamicin sulfate in water, dissolving sodium lauryl sulfate in water, and reacting the two solutions.

The coating may be applied using techniques familiar to those in the art, such as a dip coating technique from a solvent based system consisting of tetrahydrofuran (THF), ethyl alcohol, and benzyl alcohol, and other suitable techniques. In this manner, the gentamicin lauryl sulfate is entrapped in the thin coating applied to the device surfaces so that the pharmacological coating provides a diffusion barrier that allows the pharmacological agent to be released into the surrounding tissue 120 over a period of at least 10 weeks.

After dip coating the sleeve 110, the sleeve 110 may be dried in an oven. The dried sleeve 110 is packaged in a clamshell that protects the coated surface and minimizes contact of the coated surface with other packaging materials. The clamshell package then can be packaged in a foil pouch that has a gas permeable window. The foil pouch may be sterilized using gas sterilization methods familiar to those in the art. Gas sterilization allows the gas to penetrate the gas permeable window in the foil pouch. After sterilization, the foil pouch may be sealed below the window and the window is cut off. This process results in a sleeve 110 coated with pharmacological coating and sealed in a completely non permeable foil pouch, which gives the sleeve 110 a longer shelflife.

In an alternative embodiment, an antibiotic is distributed throughout at least a portion of the polymer material of the sleeve 110. In another embodiment, an antibiotic is distributed throughout the polymer material of the sleeve 110, and all surfaces of the sleeve 110 are coated with a pharmacological coating as previously described. The pharmacological coating provides effective concentrations of antibiotic at the device surface, but does not produce high systemic concentrations of antibiotic, alleviating the typical side effects associated with systemic antibiotic therapy. In alternative embodiments, the sleeve 110 may be coated with anticoagulants, antiinflammatory agents, analgesics, antihistamines or any other suitable pharmacological agent that may reduce complications associated with external fixation of bone.

In a preferred embodiment of a pharmacological sleeve 110 according to this invention, the polymer material of the sleeve 110 is extruded along the inner surface 116 of the sleeve 110 to produce at least one internal spline 114. Alternatively, the internal spline 114 may not form a part of the sleeve 110, but may be a separate structure on the inner surface 116 of the sleeve 110. The internal splines 114 may be of varying or identical size.

“Spline” as used herein means any spacer or internal spline 114 that causes the sleeve 110 or portions of the sleeve 110 to be distanced from an external fixation element 118 or member. With the exception of creating and maintaining distance between the sleeve 110 and an external fixation element 118, a spline 114 need not, and preferably does not, limit rotation or any other motion of the sleeve 110 relative to an external fixation element 118.

In one embodiment of the invention, the internal splines 114 are longitudinal splines 114 and extend along the entire length of the sleeve 110. In another embodiment, the longitudinal internal splines 114 extend along a portion of the length of the sleeve 110. In alternative embodiments, the internal splines 114, shown in FIGS. 3 and 4, may be triangular 130, square 132, semicircular 134, or semihexagonal or any other suitable shape to facilitate contact with external fixation elements 118, which are circular, hexagonal, or any other multisided shape. In a preferred embodiment, shown in FIG. 4, a pharmacological sleeve 110 includes three internal splines 114 that are spaced 120° apart. In another embodiment, a pharmacological sleeve 110 includes one internal spline 114 that forms a helix on the inner surface 116 of the sleeve 110.

As shown in FIGS. 3 and 4, the internal splines 114 of the pharmacological sleeve 110 create a space 126 between the inner surface 116 of the pharmacological sleeve 110 and the outer diameter of the external fixation element 118. The space 126 provides for wound drainage around the entire external fixation element 118. The space 126 also provides an access route for the delivery of drugs directly into the wound site. Drugs may be administered via a syringe needle into the space 126 between the pharmacological sleeve 110 and the external fixation element 118, which helps prevents the accumulation of bacteria at the wound site and the formation of bacterial biofilm on the external fixation element 118.

The pharmacological coating provides a diffusion barrier that allows the prolonged release of drugs into the space 126 and a bactericidal level of drugs may collect in the space, preventing adhesion of bacteria to the metal surface of the external fixation element 118 and subsequent formation of biofilm on the external fixation element 118.

In one embodiment, the pharmacological coating slowly releases gentamicin from the pharmacological sleeve 110 surfaces over a period of 26 weeks. The gentamicin may accumulate at both the interface of the sleeve 110 and tissue 120 and within the space 126 between the pharmacological sleeve 110 and metal external fixation element 118. This accumulation of drugs at the wound site effectively prevents bacterial adhesion to the fixation element and bacterial accumulation in tissues 120 around the wound site.

The internal splines 114 allow for more effective release of drugs from the pharmacological coating. The release of drugs from the pharmacological coating on the inner surface 116 of the sleeve 110 has been shown to increase the overall drug elution from pharmacological sleeves 110. In an experiment comparing the cumulative drug elution from the pharmacological coating on pharmacological sleeves 110 with internal splines 114 to the cumulative drug elution from the pharmacological coating on pharmacological sleeves 110 with smooth inner surfaces, the pharmacological sleeves 110 with internal splines 114 eluded 50-75% more gentamicin than the pharmacological sleeves 110 with smooth inner surfaces. This increase in drug elution provides increased efficacy of pharmacological sleeves 110 with internal splines 114.

Internal splines 114 provide an improved fit around external fixation elements 118 as illustrated by FIGS. 3-6. Further, internal splines 114 provide tolerance between mismatched external fixation elements 118 and pharmacological sleeve 110 sizes because the polymer between the internal splines 114 is able to bend to accommodate a slightly larger external fixation element 118. In this manner, minor variations in pharmacological sleeve 110 internal diameters may be accommodated and the sleeve 110 is easier to apply around an external fixation element 118 during surgery. Consequently, the pharmacological sleeve 110 is less expensive to manufacture because one pharmacological sleeve 110 having internal splines 114 is able to fit external fixation elements 118 having a wider range of diameters.

Internal splines 114 also provide an inflection point for the pharmacological sleeve 110. External stresses applied on the pharmacological sleeve 110 can be absorbed by the polymer between internal splines 114, while the internal splines 114 maintain a friction grip on the surface of the external fixation element 118. This stress absorption can accommodate the tissue swelling that is part of the healing process and will prevent movement and extrusion of the pharmacological sleeve 110 by the wound site.

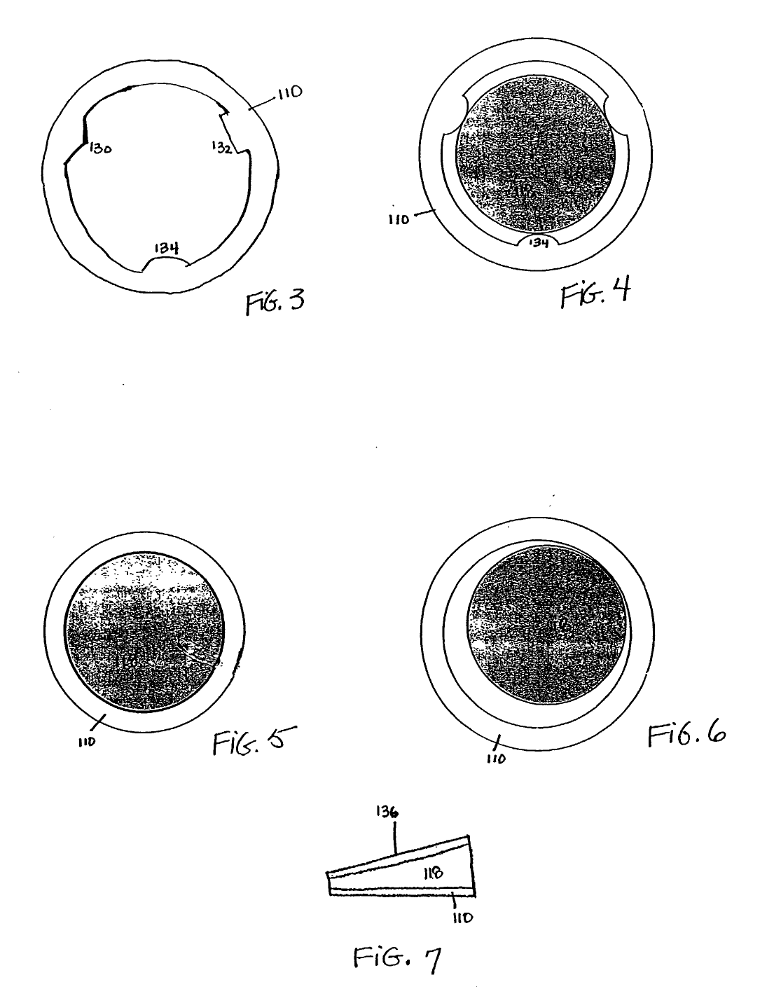

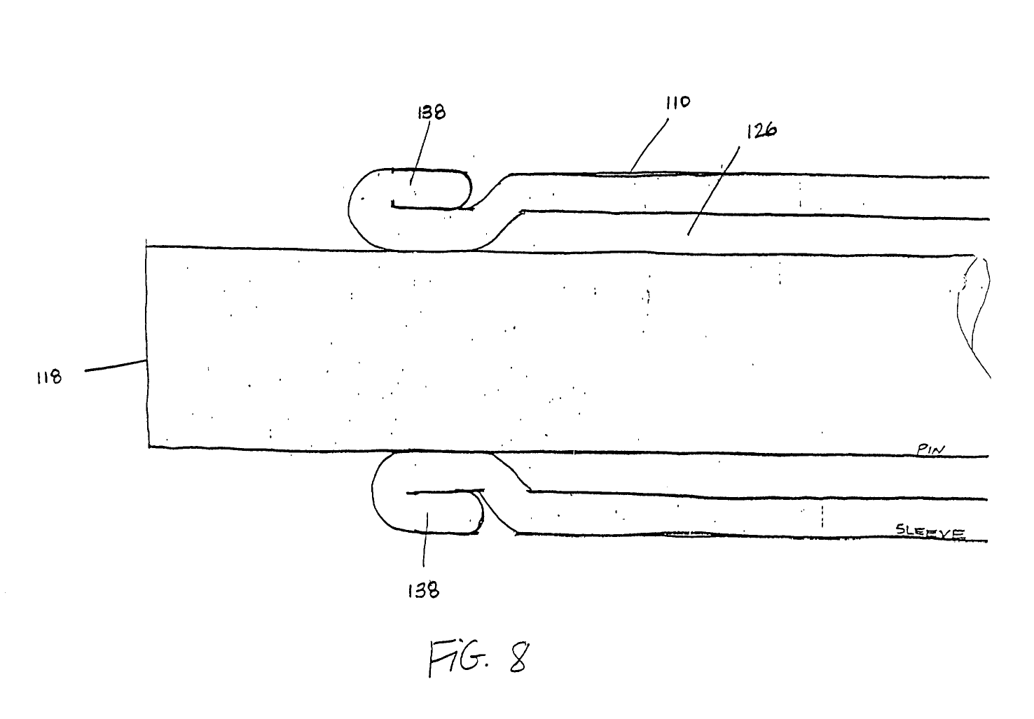

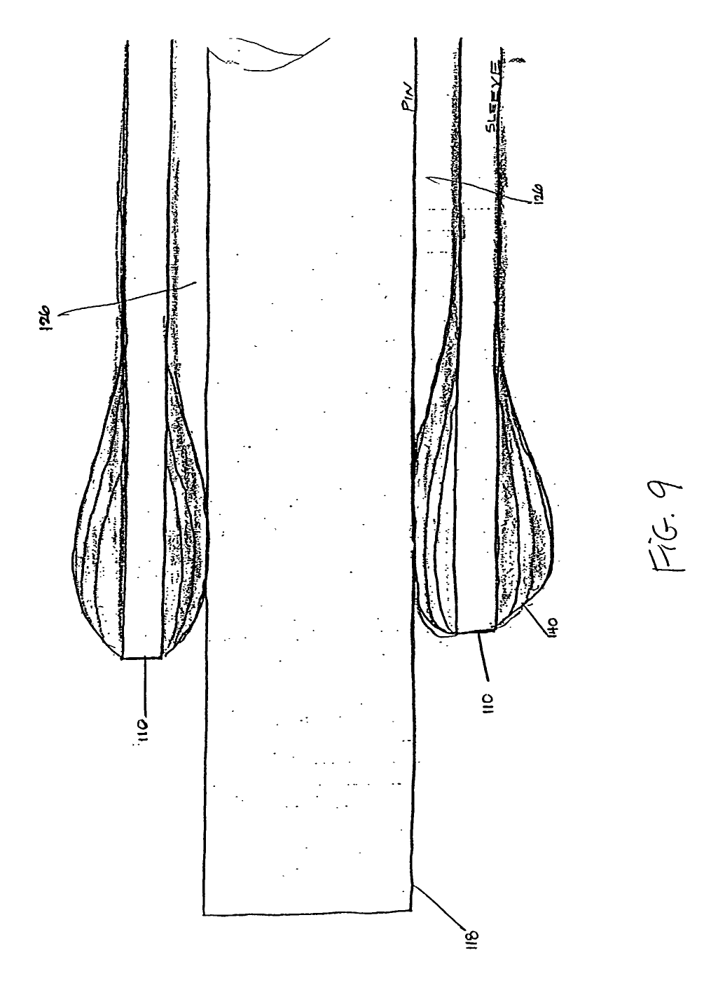

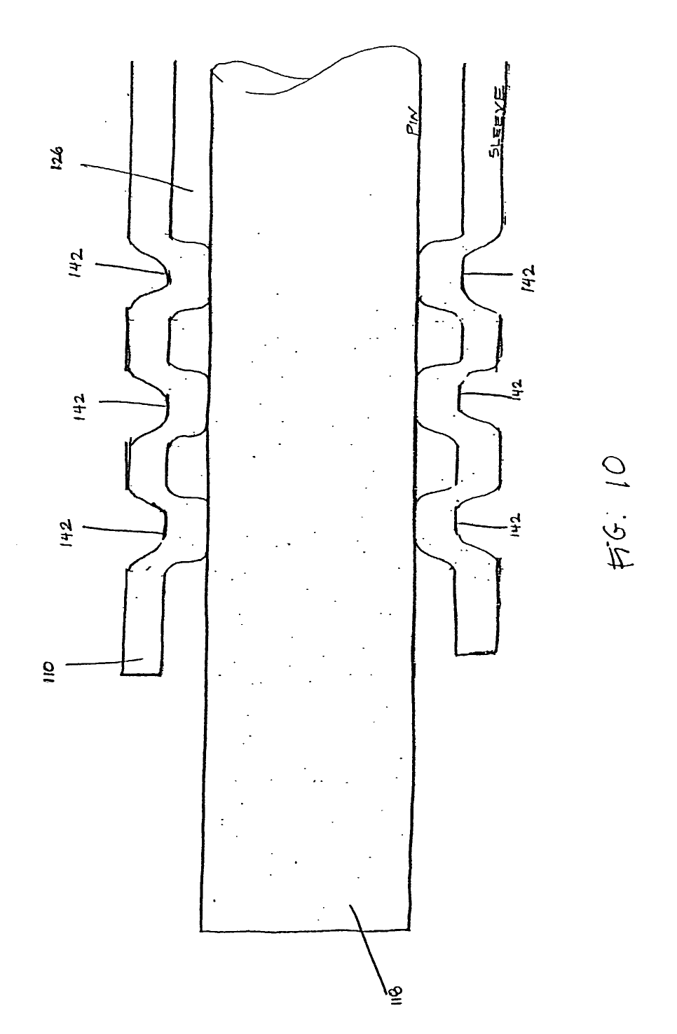

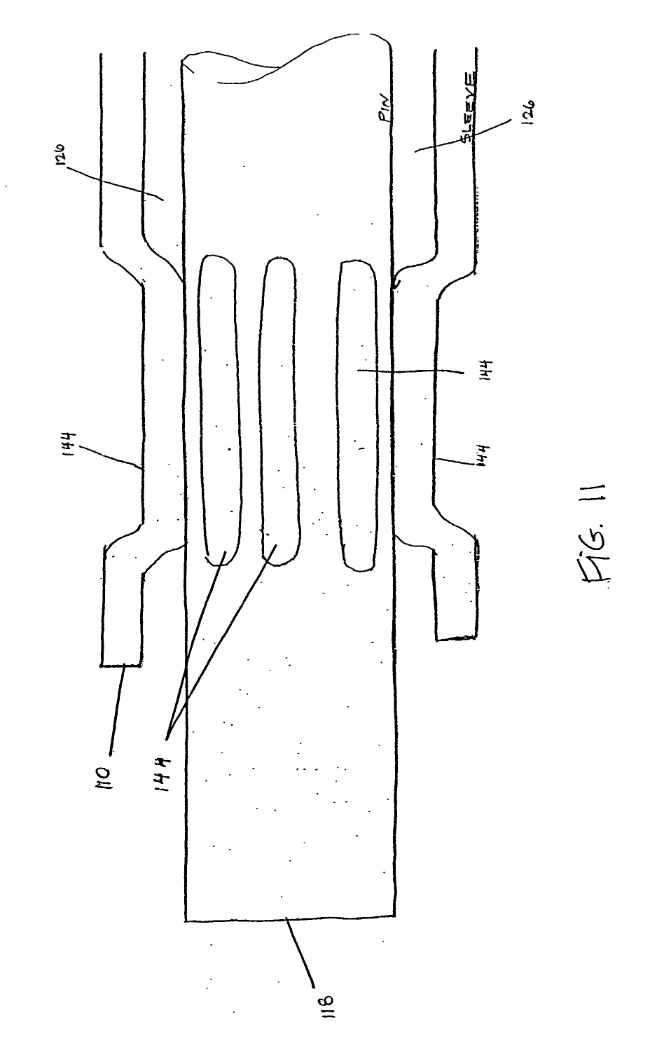

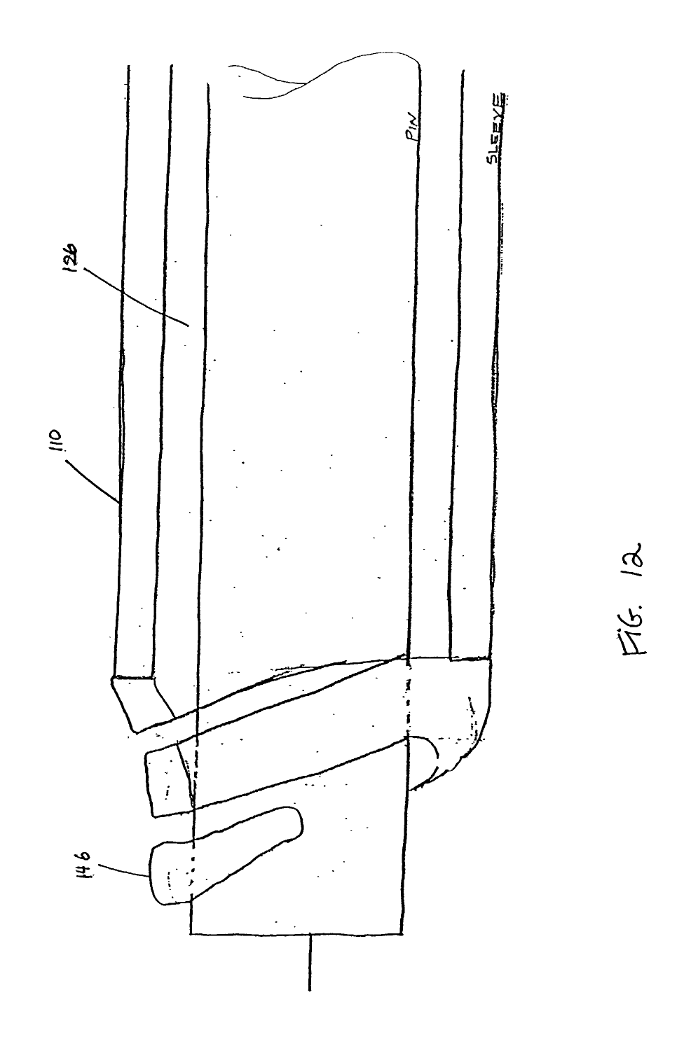

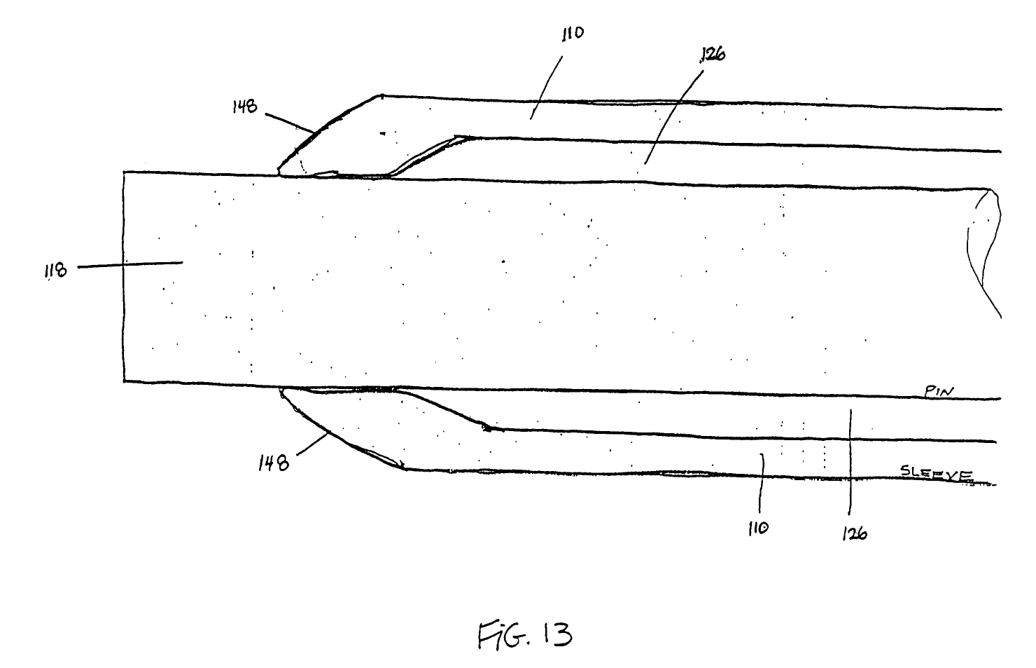

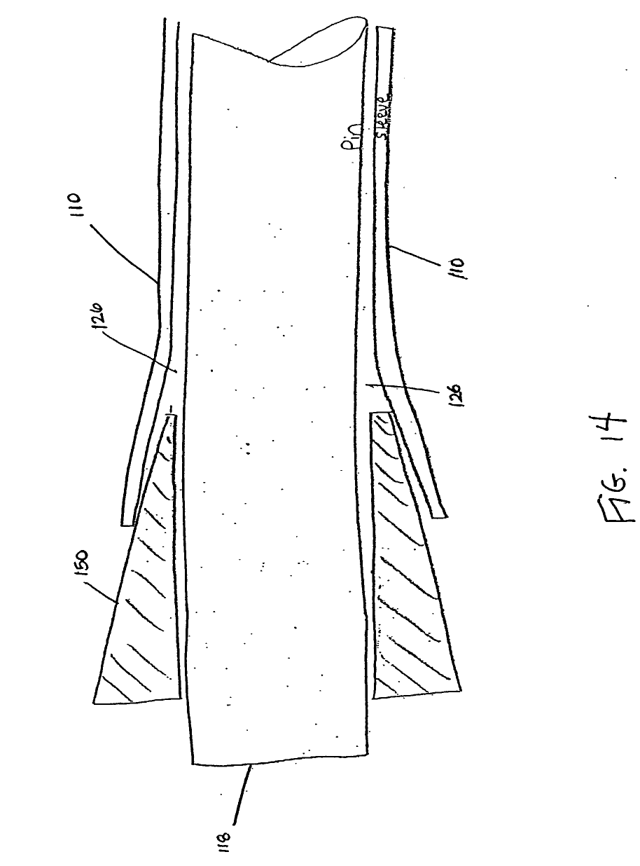

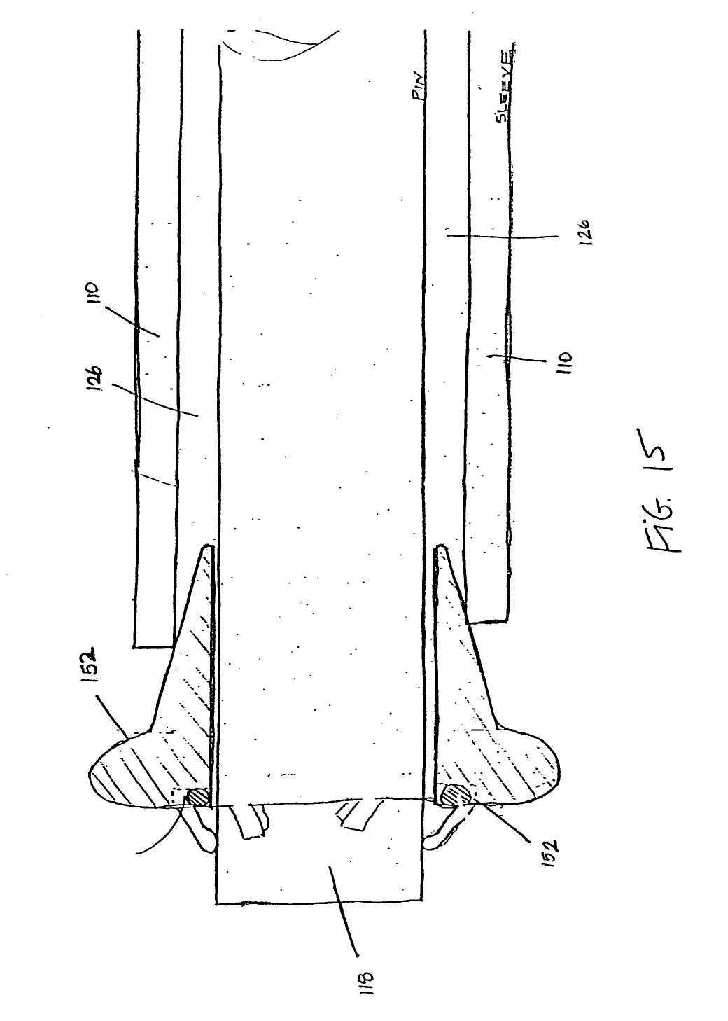

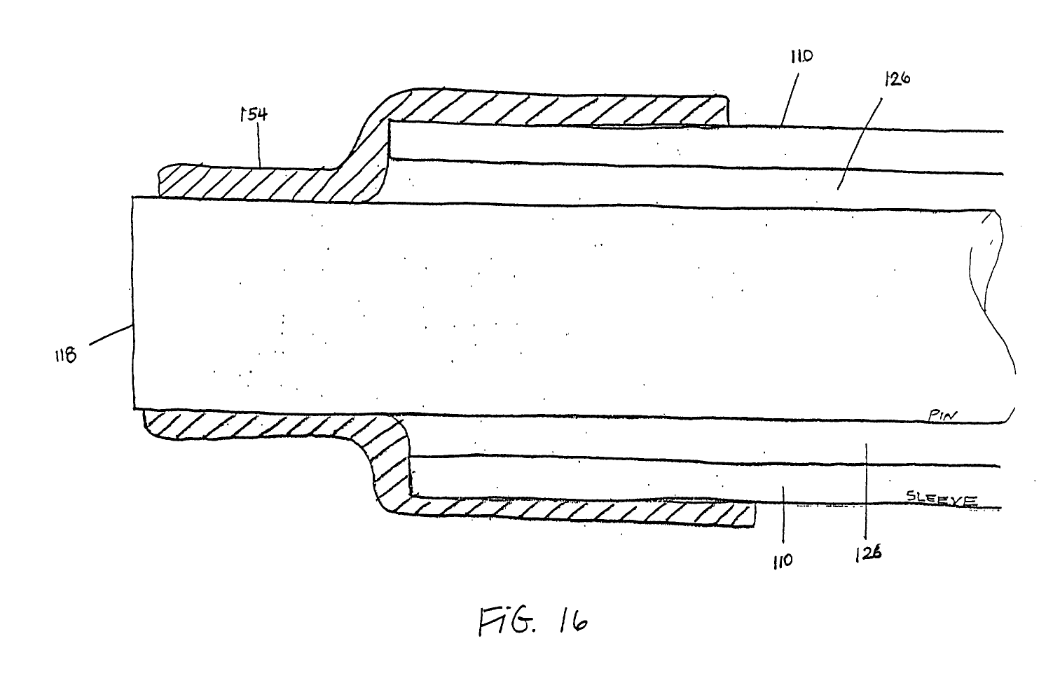

A pharmacological sleeve 110 according to this invention may be positioned on an external fixation element 118 with a positioning mechanism 124. In one embodiment, a pharmacological sleeve 110 includes an integrated positioning mechanism 124 on the proximal end of the sleeve 110 that secures the sleeve 110 in position on the external fixation element 118. As shown in FIGS. 7-13, the integrated positioning mechanism 124 may include a heat shrink taper 136, a rolled edge 138, excess coating 140, an indentation 142, crimps 144, a pigtail 146, and a heat shrinkable nose cone 148.

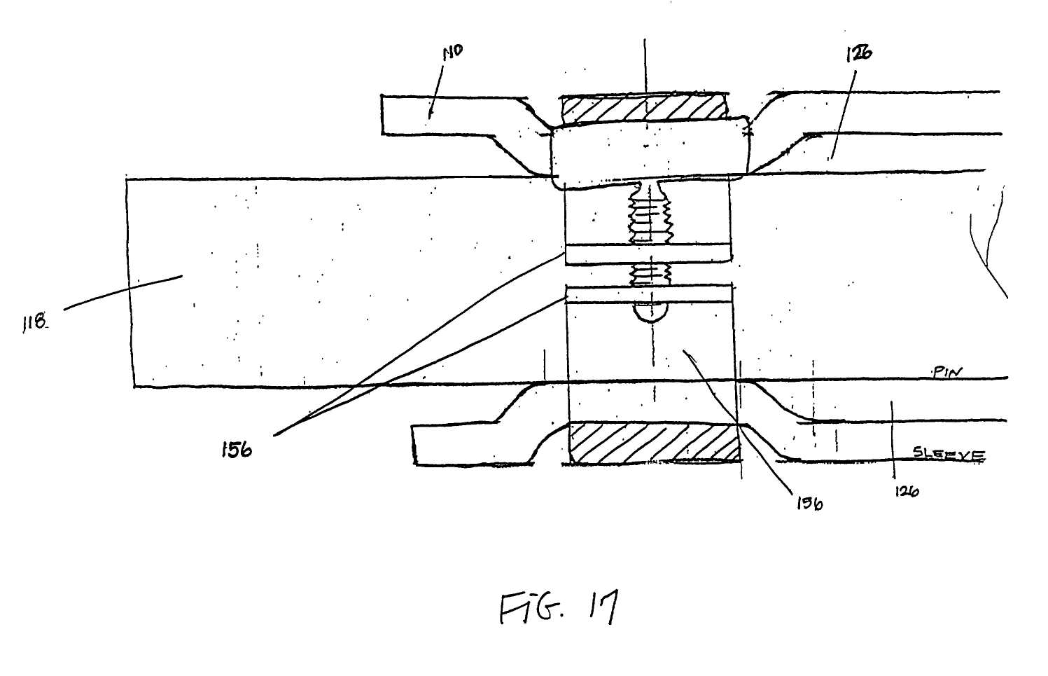

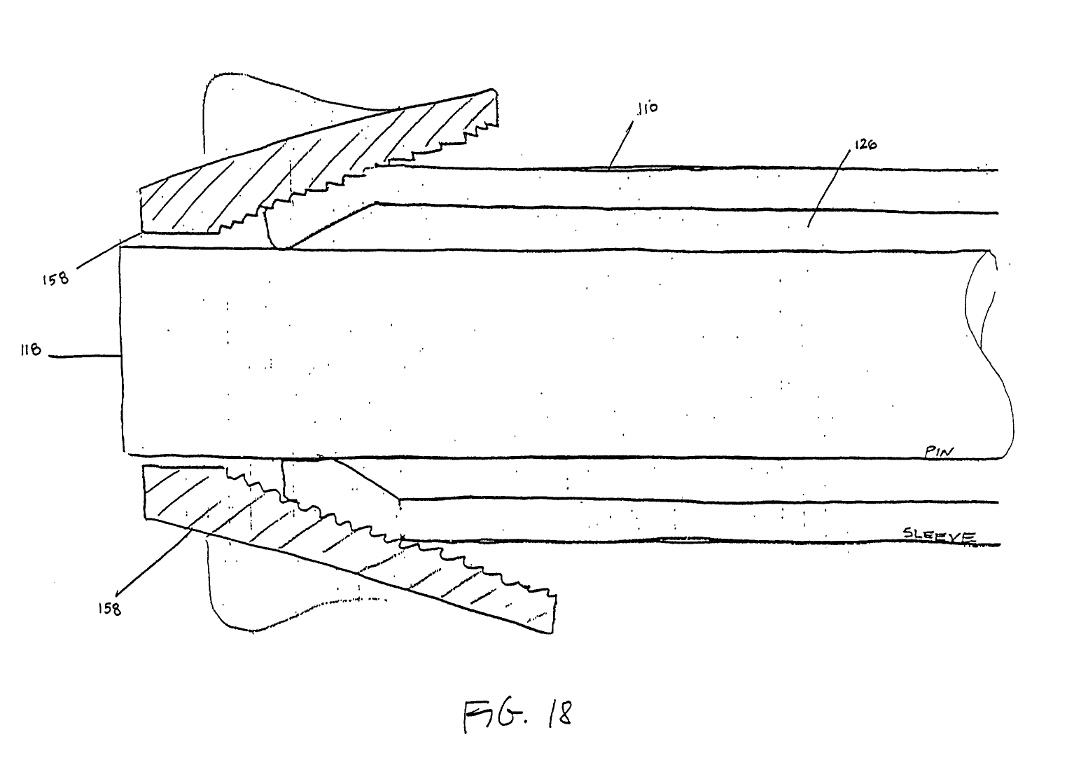

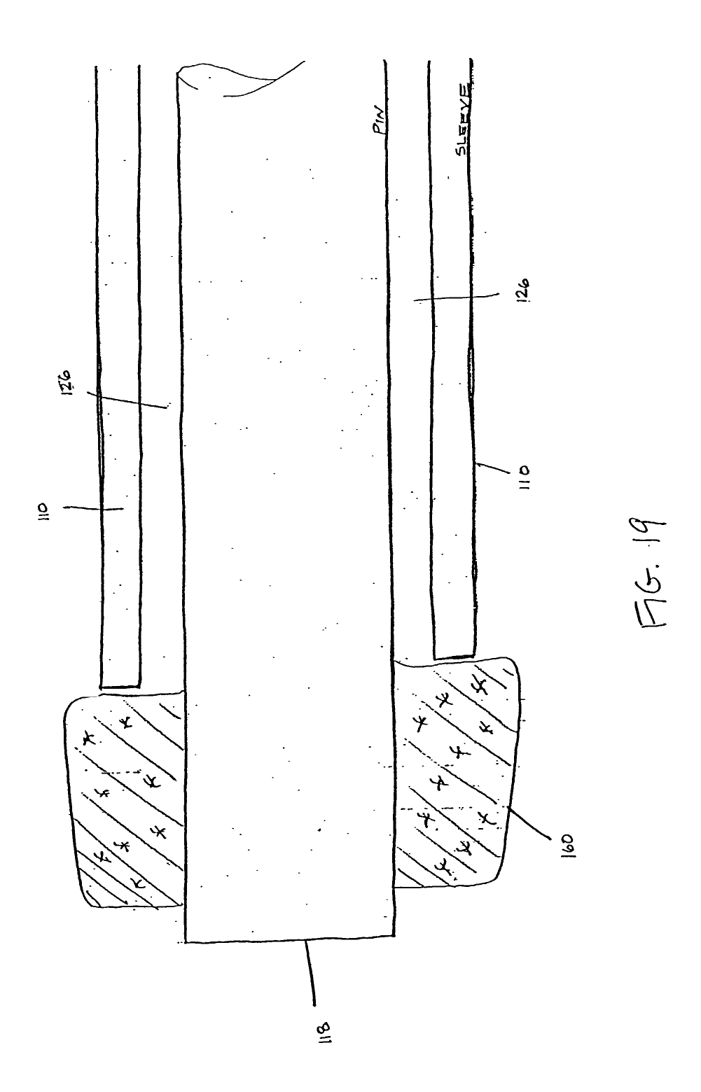

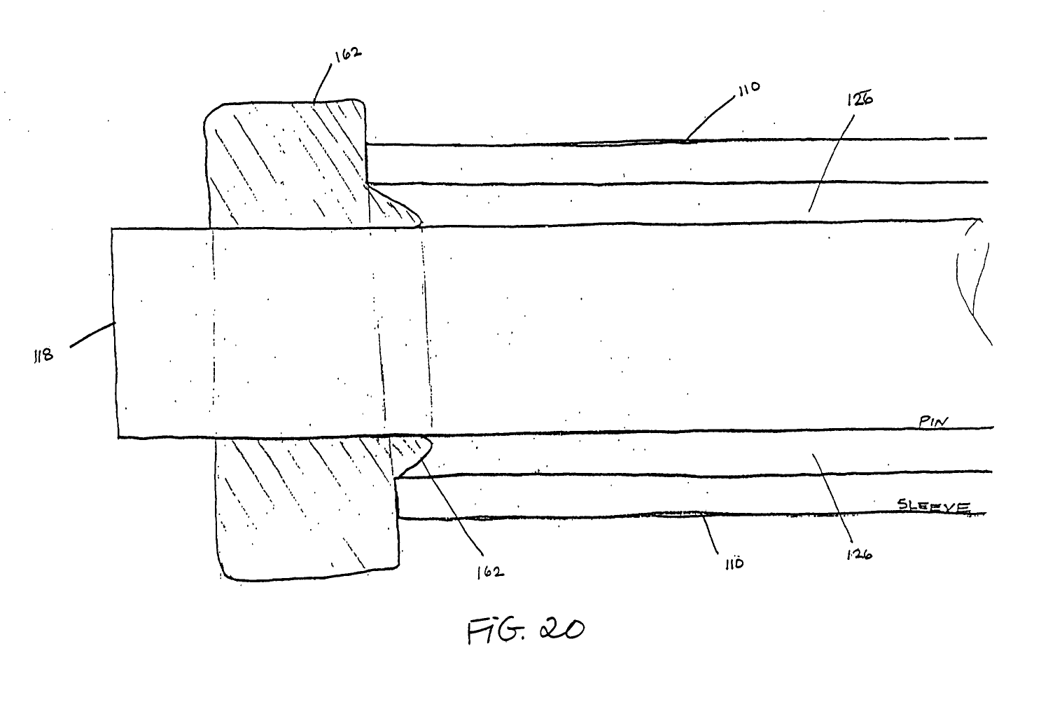

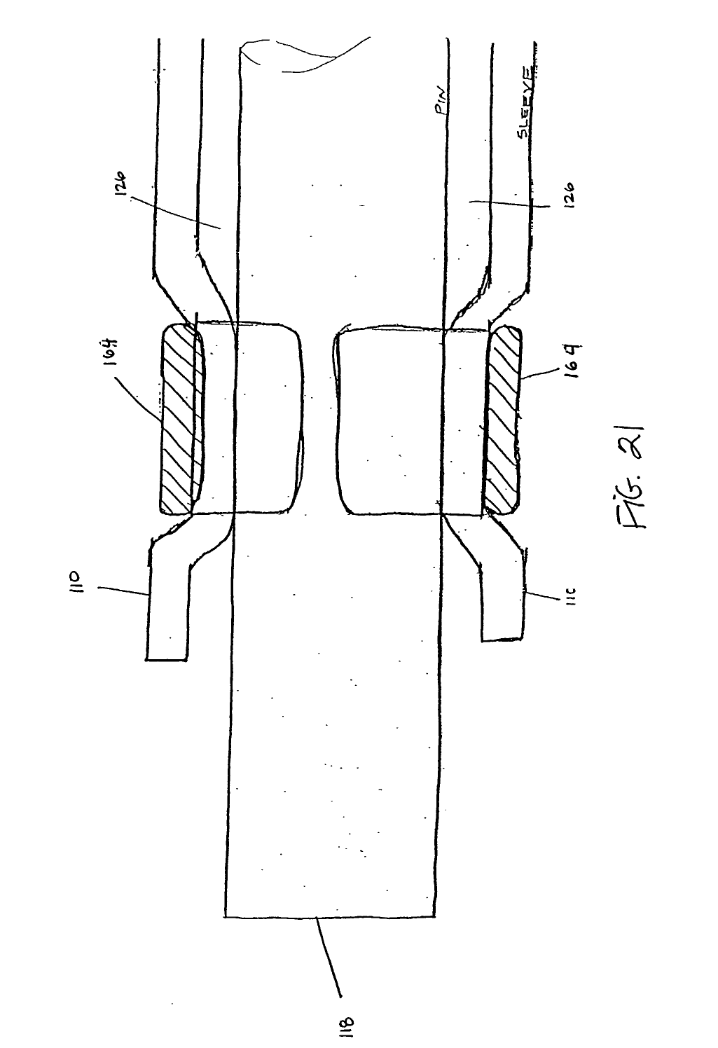

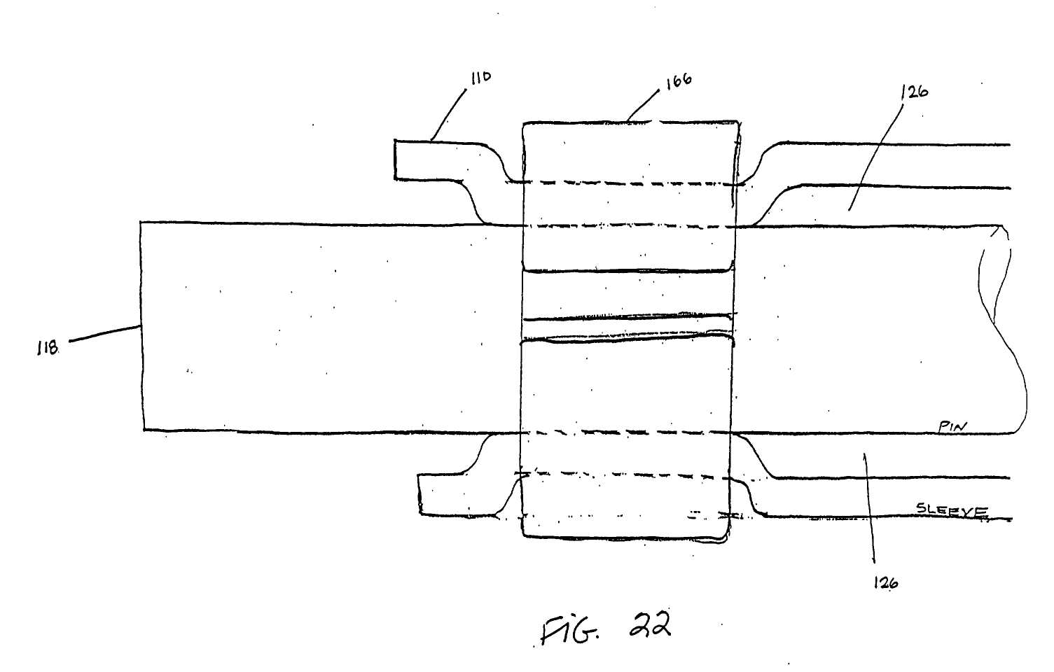

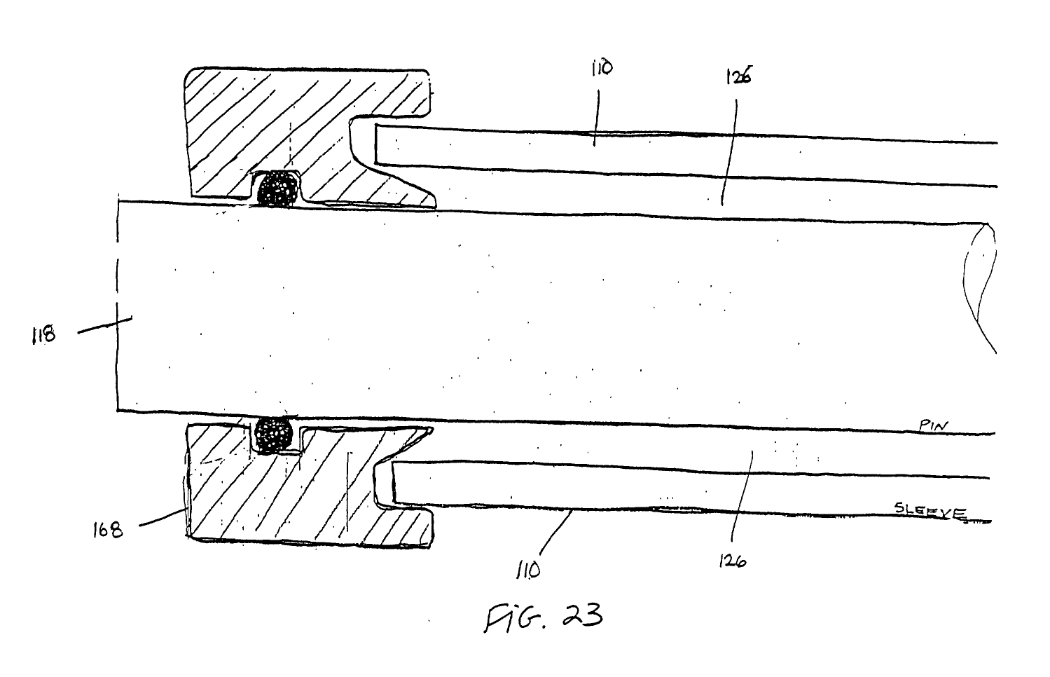

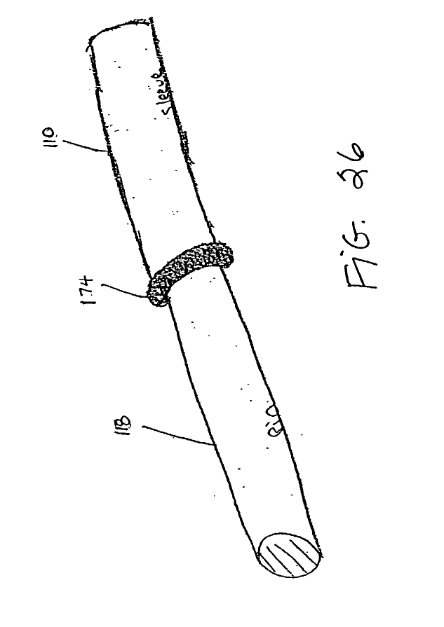

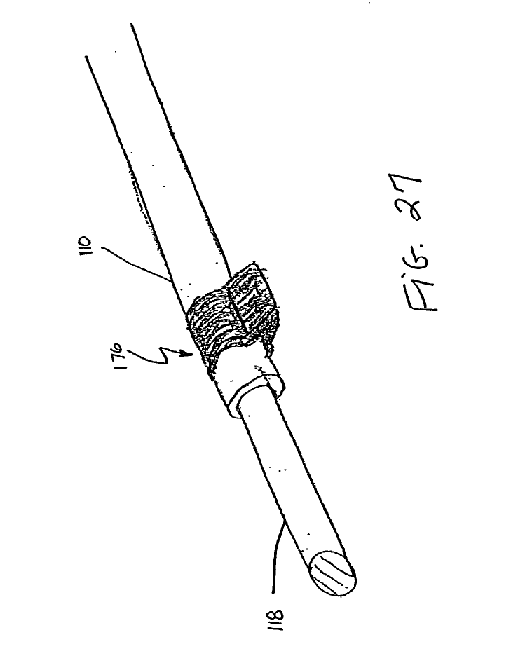

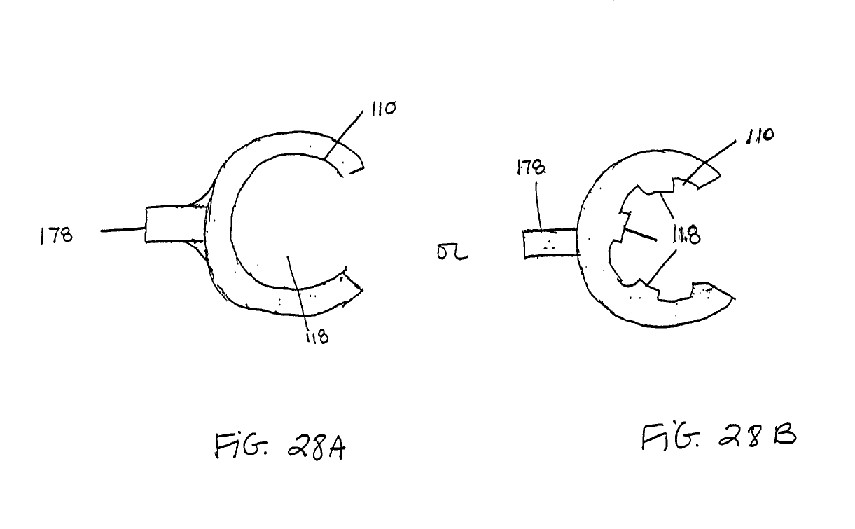

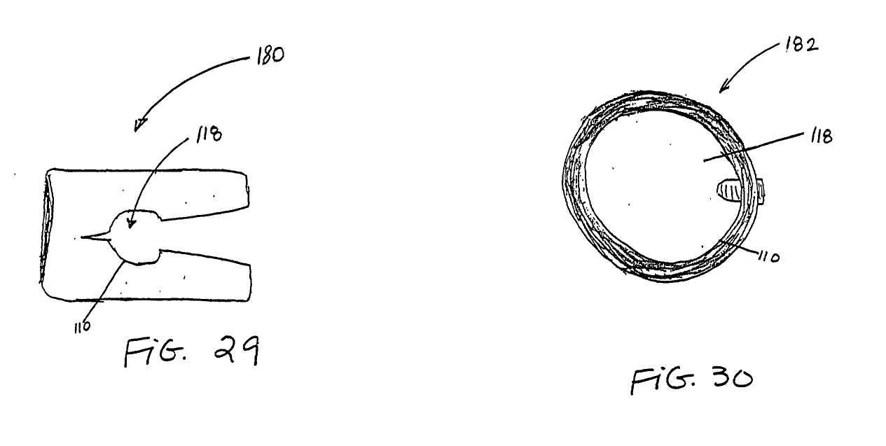

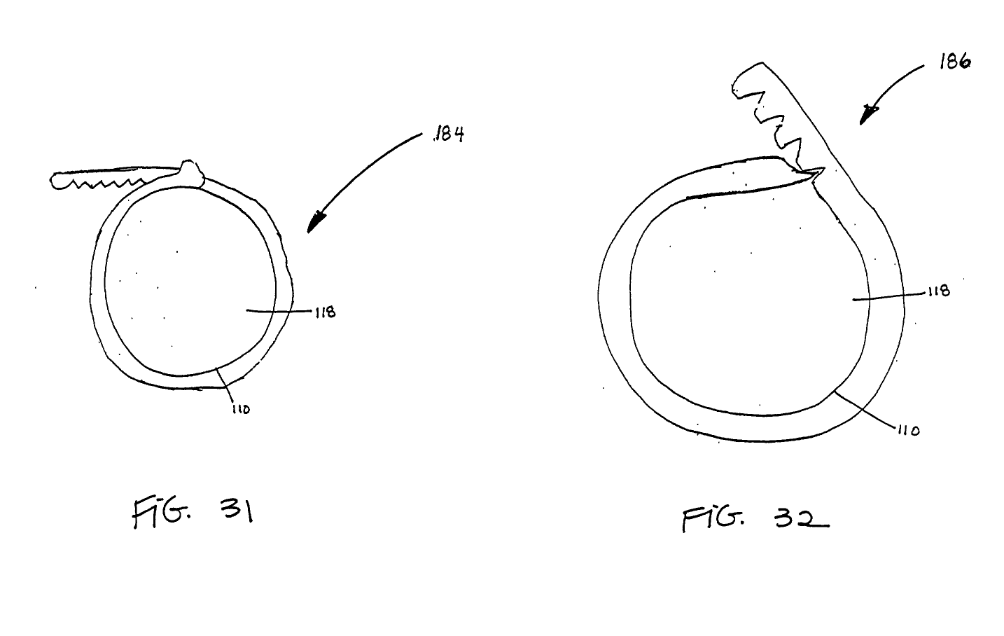

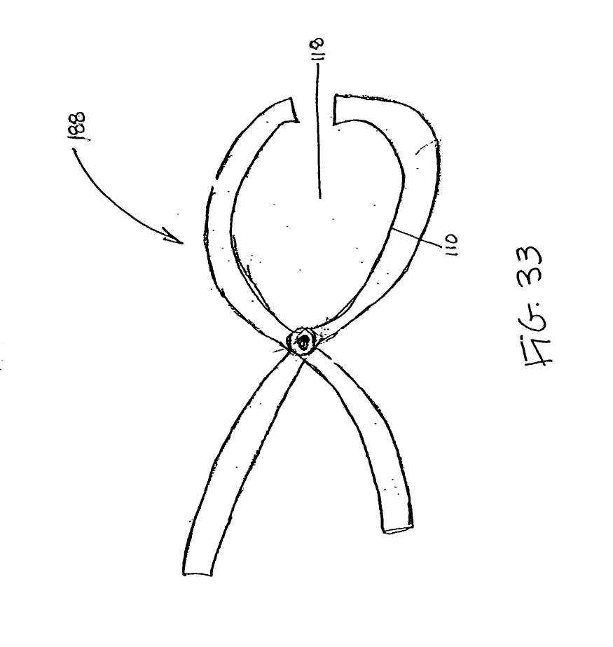

In another embodiment, a pharmacological sleeve 110 may be positioned on an external fixation element 118 with a removable or adjustable positioning mechanism 124. As shown in FIGS. 14-33, the removable or adjustable positioning mechanism 124 may include a wedge 150, a retainer ring 152, heat shrink tubing 154, a hose clamp 156, a tapered connector 158, a cap 160, a cap with a tapered seal 162, a retainer band 164, a toothed hinge clamp 166, a cap with an O-ring 168, a clip with two interlocking arms 170, a snap-on clip 172, an O-ring 174, a clip with a V-shaped body 176, a C-clamp 178, a disk clip 180, a set screw clamp 182, a toothed clamp 184, a toothed clip 186, and a chip or spring loaded hinge clip 188. Several integrated positioning mechanisms, such as the rolled edge 138, excess coating 140, and heat shrinkable nose cone 148, and several removable or adjustable positioning mechanisms, such as a wedge 150, a retainer ring 152, heat shrink tubing 154, a hose clamp 156, a tapered connector 158, a cap 160, a cap with a tapered seal 162, a retainer band 164, a toothed hinge clamp 166, a cap with an O-ring 168, a clip with two interlocking arms 170, a snap-on clip 172, an O-ring 174, a toothed clamp 184, and a toothed clip 186, may be used to seal the space 126 between the external fixation element 118 and the pharmacological sleeve 110.

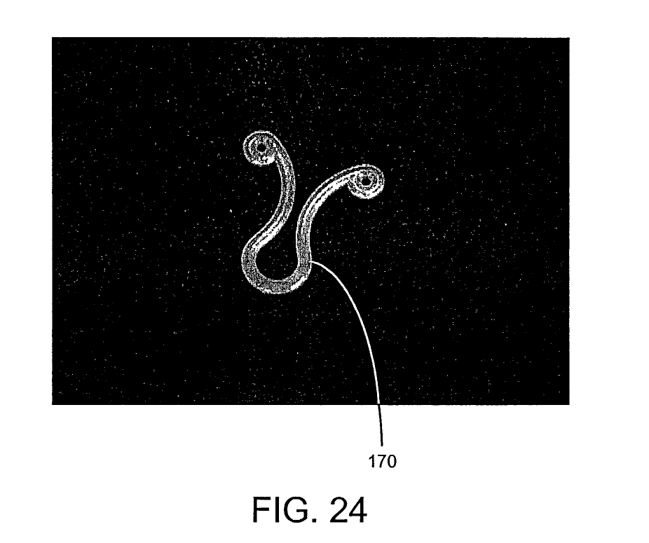

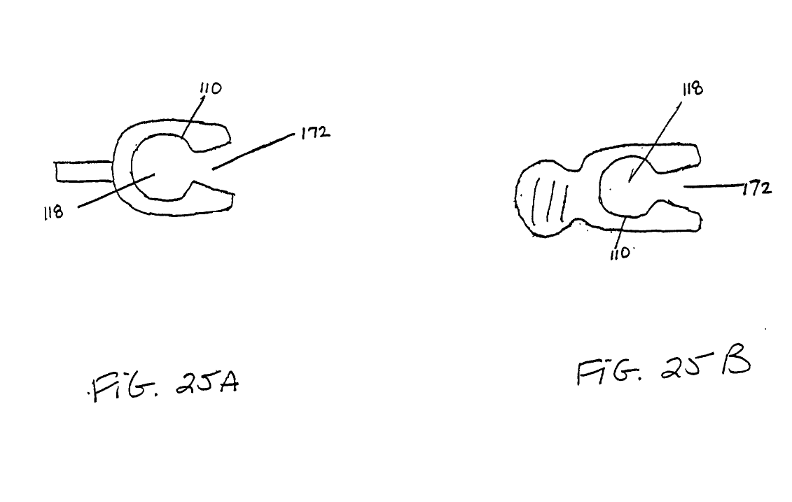

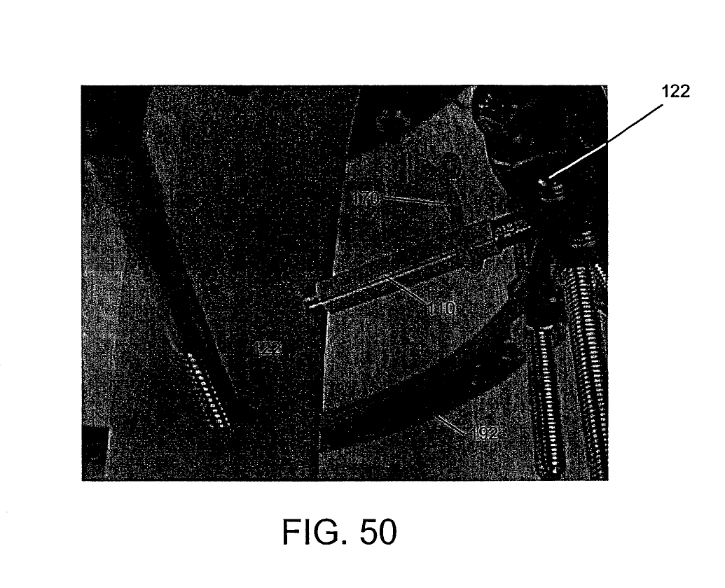

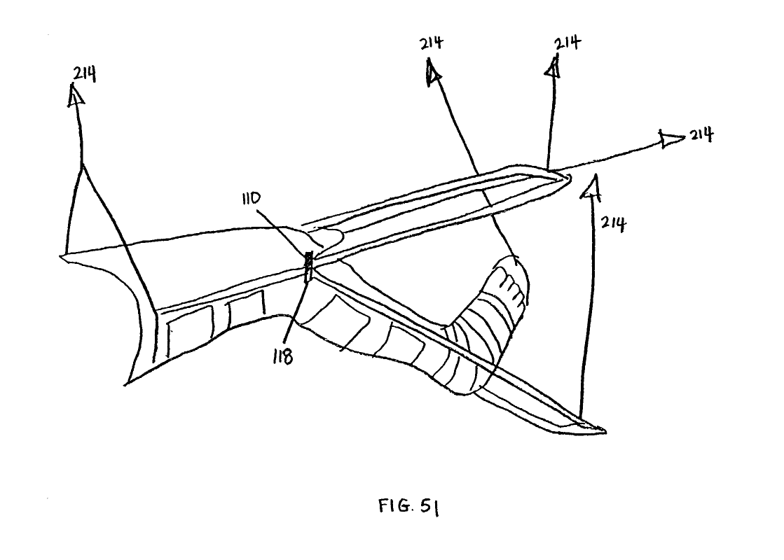

In a preferred embodiment, a pharmacological sleeve 110 is positioned on an external fixation element 118 with a clip with two interlocking arms 170 that twist around each other to hold the sleeve 110 in place, as shown in FIGS. 24 and 50. In one embodiment, the clip 170 is composed of a nylon material. In alternative embodiments, the clip 170 may be composed of plastics, polymers, rubber or other flexible materials. The clip 170 maintains a cylindrical cross-section and can be easily placed around the sleeve 110 positioned on the external fixation element 118. Alternatively, the cross-section of the clip 170 may be square, rectangular or any other shape. If desired, the clip 170 can be easily removed after it has been locked in place by twisting the two arms apart. When installed on a sleeve 110, an inside diameter of the clip 170 is within the range between an outside diameter of a pin and an outside diameter of the sleeve 110.

In another embodiment, a pharmacological sleeve 110 is not secured by a positioning mechanism 124, which promotes more effective wound drainage around the external fixation element 118.

In one method of using a pharmacological sleeve 110 according to one embodiment of this invention, the external fixation element 118 is attached to fractured or malformed bone 122. The process of attaching an external fixation element 118 to a bone 122 includes first making an incision in the skin surface and in the subcutaneous fat and muscle tissues 120 surrounding the bone 122. The bone 122 is then tapped to receive the threaded end of the external fixation element 118. The external fixation element 118 is attached to the bone 122 by inserting the threaded end of the external fixation element 118 into the incision and screwing it into the tapped cavity located within the bone 122. Alternatively, the external fixation element 118 may be self-tapping, whereby the external fixation element 118 is attached to the bone 122 by first drilling a cavity in the bone 122 and then screwing an external fixation element 118 into the cavity. Still yet, the external fixation element 118 may be selfdrilling, whereby the external fixation element 118 is attached to the bone 122 by simply screwing it into the bone 122.

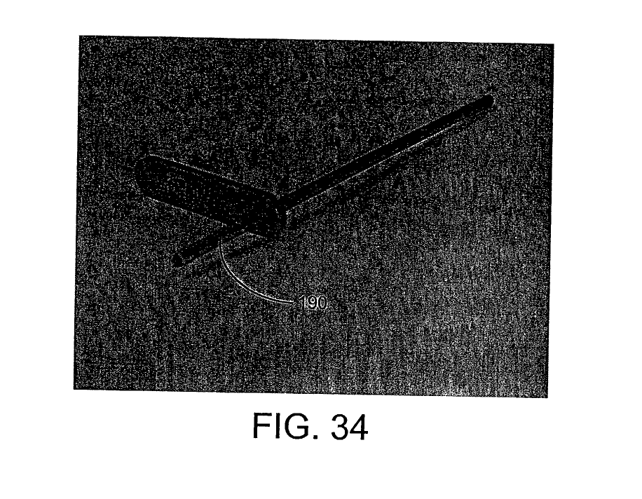

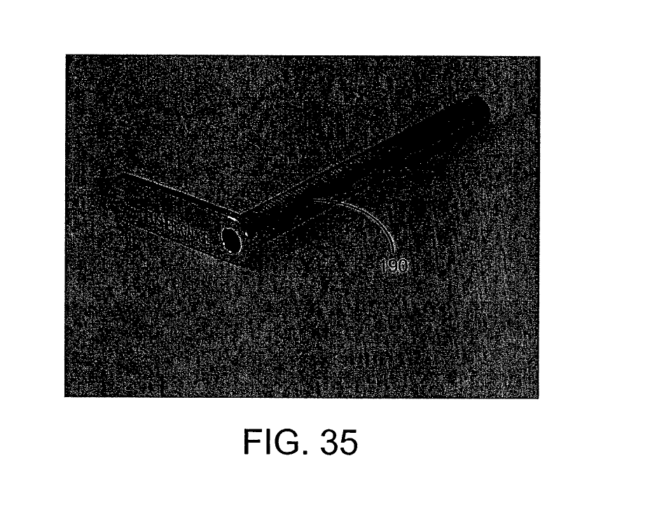

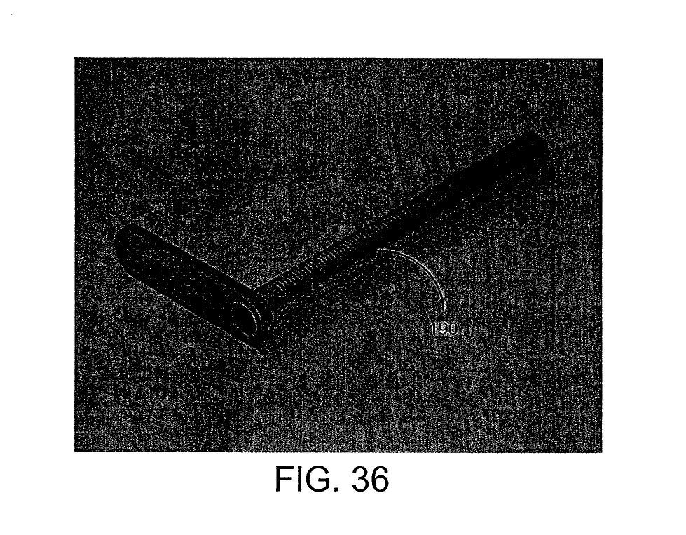

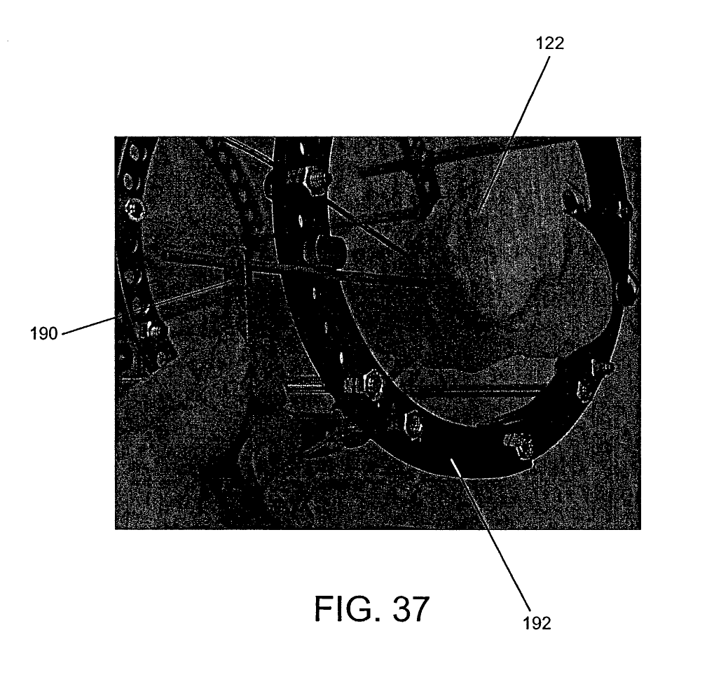

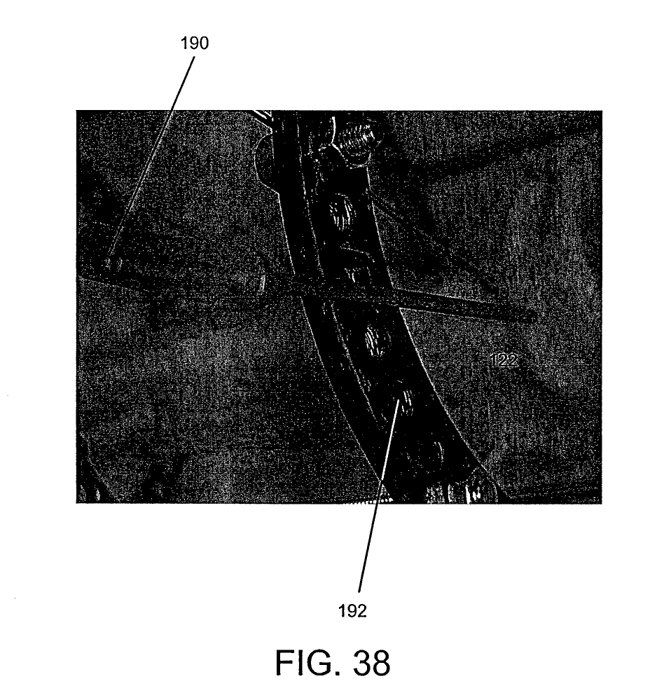

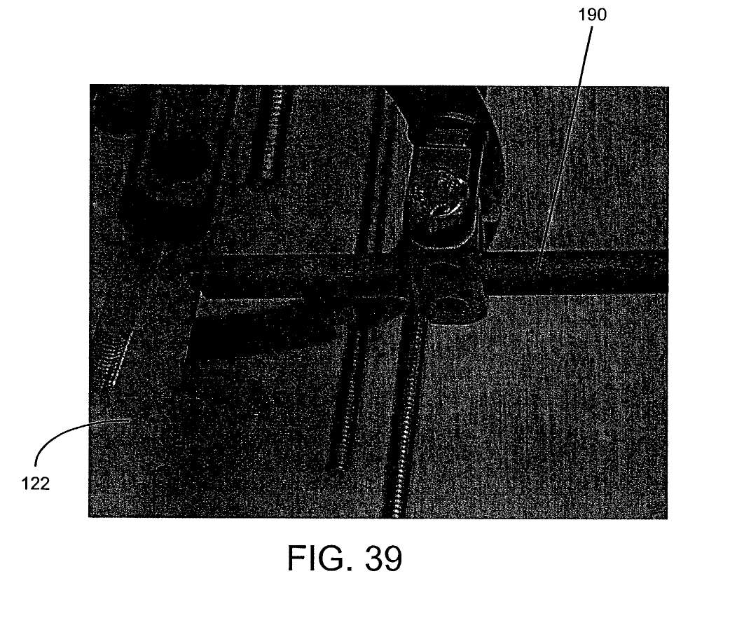

Once the external fixation element 118 has been attached to the bone 122, the surgeon may select a gauge/pusher 190 having the same diameter as the external fixation element 118, which is shown in FIGS. 34-36. As shown in FIG. 37, the gauge/pusher 190 may be slid down the external fixation element 118 to the bone 122 to release tissue 120 from around the external fixation element 118. The gauge/pusher 190 has indicia along its outer surface that should be used to determine the correct length to cut the sleeve 110, which is shown in FIGS. 38 and 39. The indicia may be etched, painted, printed, marked, or any other suitably imprinted calibrations along the outer surface of the gauge/pusher 190.

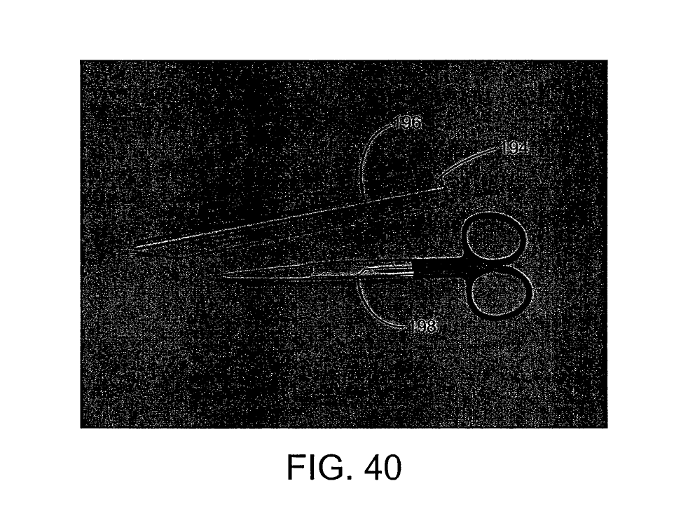

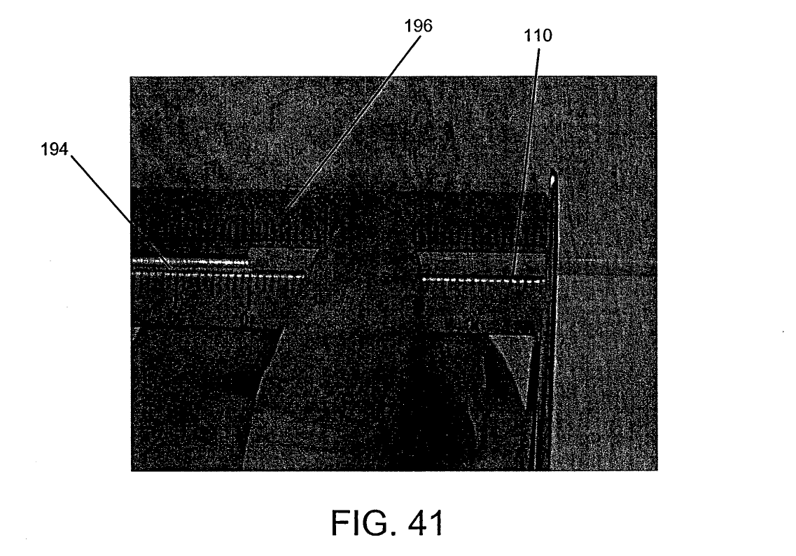

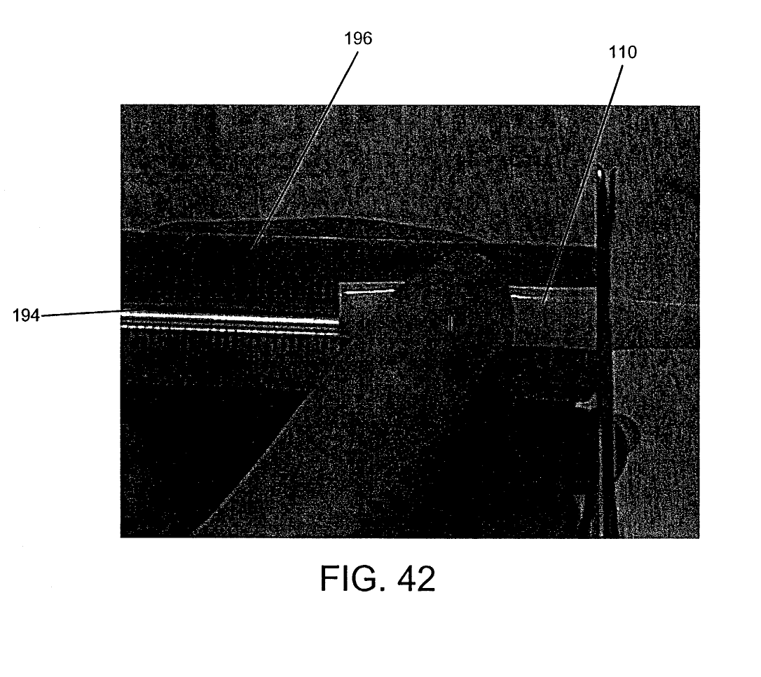

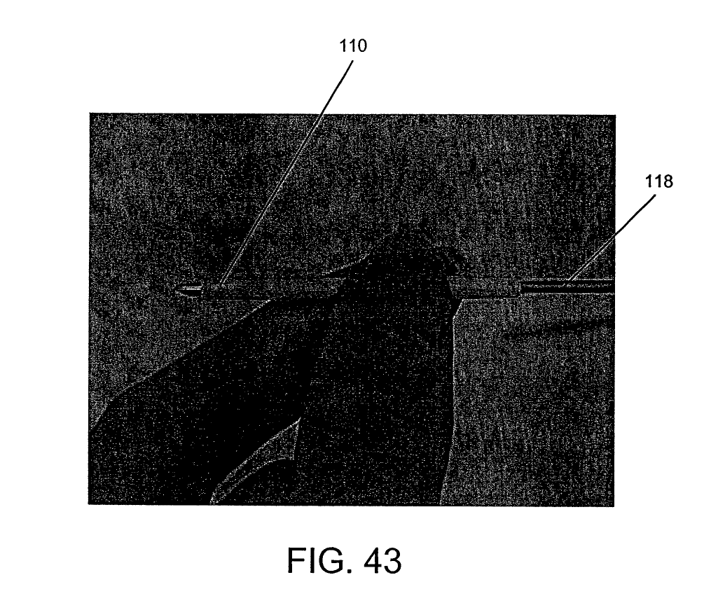

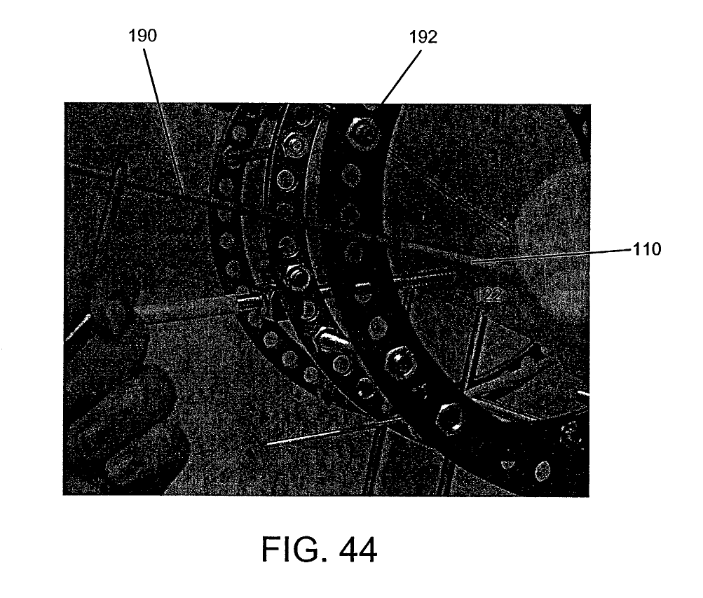

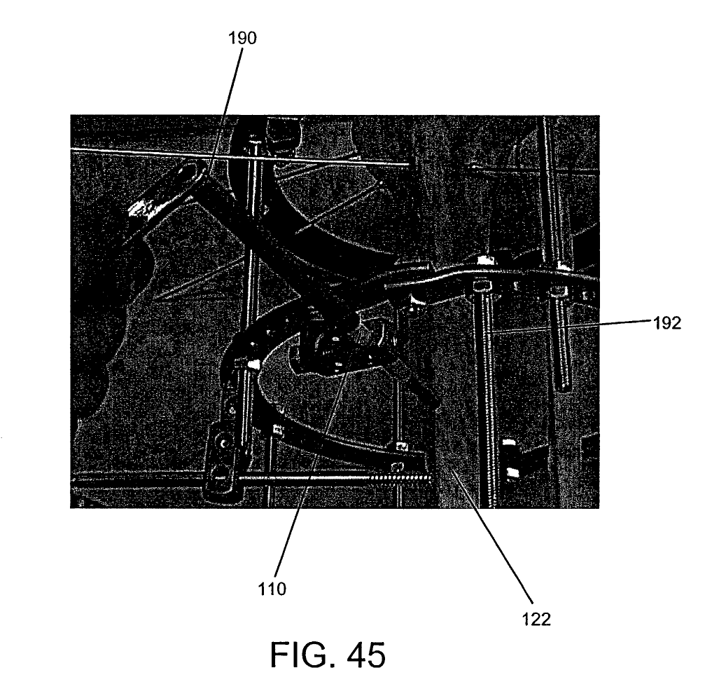

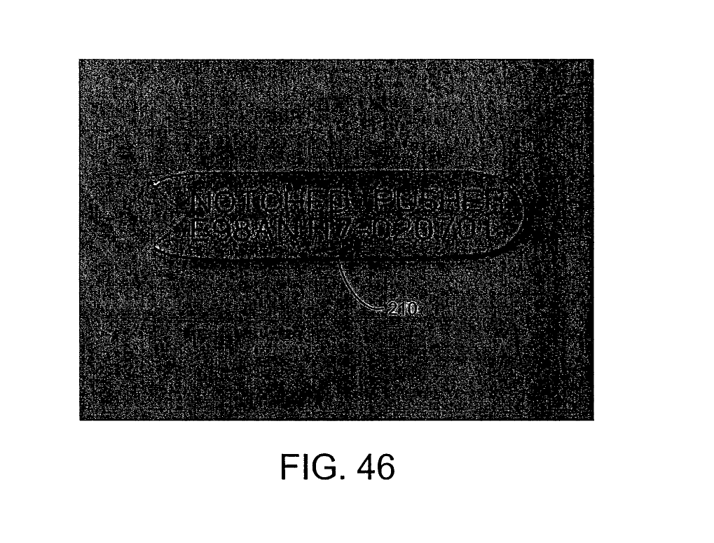

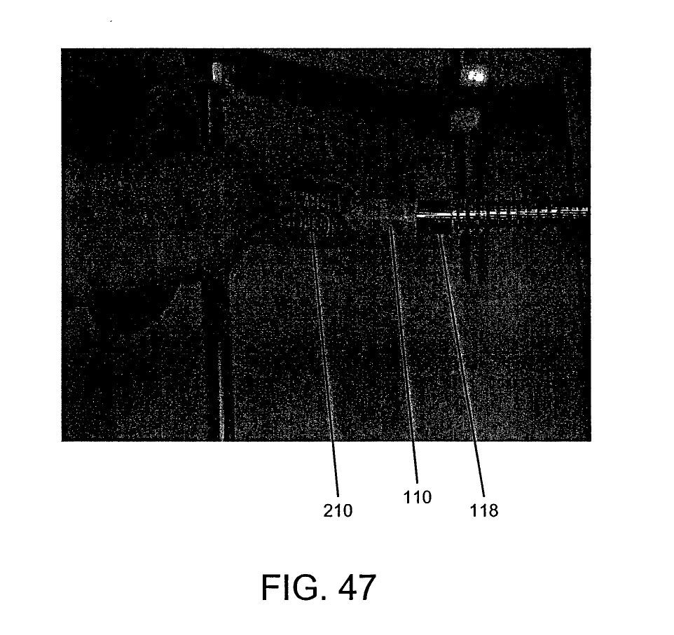

The sleeve 110 is preferably cut a sufficient length to extend from the bone 122, through the subcutaneous tissue 120, and outside of the wound a minimal distance. Too long a sleeve 110 may result in clamping the sleeve 110 in a bone fixator apparatus 192, which should be avoided. The surgeon may place the sleeve 110 in the groove 194 of a grooved ruler 196, depicted in FIG. 40, so that the sleeve 110 is aligned with the measurement taken with the gauge/pusher 190. As shown in FIGS. 41 and 42, the sleeve 110 may be held in place against the grooved ruler 196 and cut to the proper length with scissors 198. Then, the surgeon may place the correctly sized sleeve 110 onto the external fixation element 118 and the gauge/pusher 190 may be used to slide the sleeve 110 down the fixation element to the bone 122, as illustrated by FIGS. 43-45. FIG. 46 shows a notched pusher 210, which may be used to maneuver the sleeve 110 back and forth on the external fixation element 118 for positional adjustment, as shown in FIG. 47.

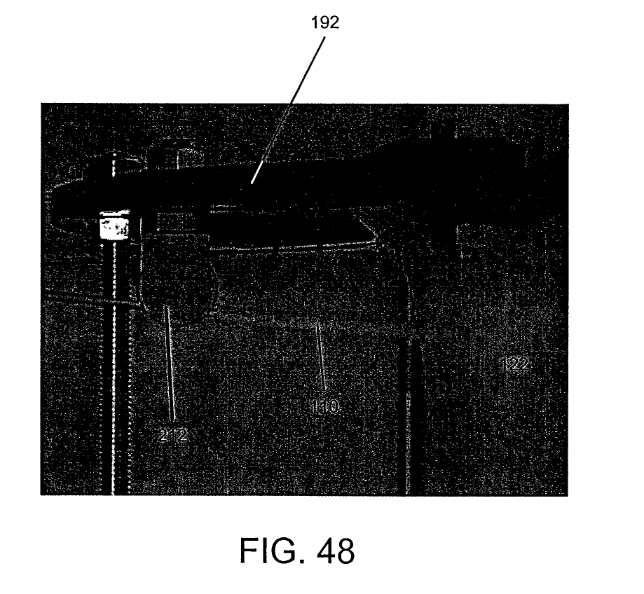

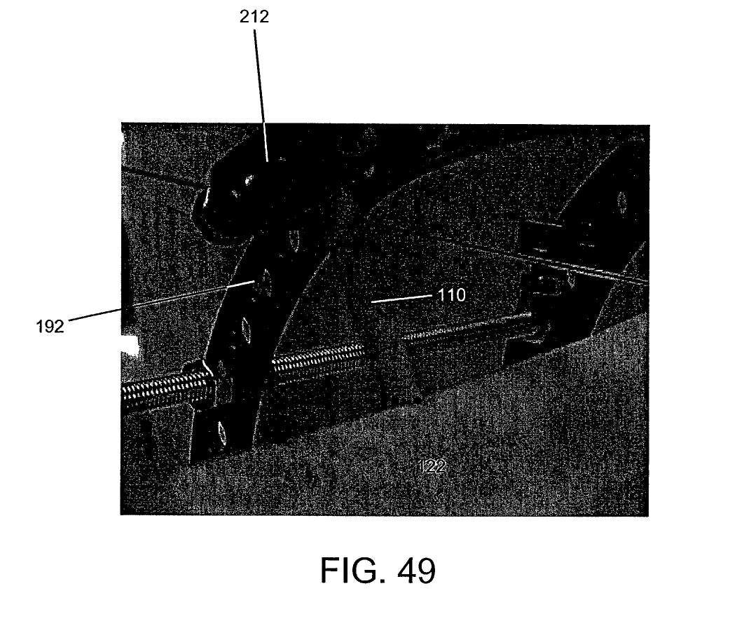

After placing the sleeve 110 on the external fixation element 118, a fastening mechanism 212, such as a nut or bolt, on a bone fixator apparatus 192 may be tightened to apply tension to the external fixation element 118, and as demonstrated in FIGS. 48 and 49, the external fixation element 118 is attached to the bone fixator apparatus 192. The bone fixator apparatus 192 may include full or half rings supported by vertical or triangular struts.

If the sleeve 110 is undersized, an integrated or a removable positioning mechanism 124, as shown in FIG. 50, may be used to hold the sleeve 110 in position on the external fixation element 118. This method may be repeated to apply sleeves 110 to other external fixation elements 118 that may be attached to a bone fixator apparatus 192.

In another method of using a pharmacological sleeve 110 according to one embodiment of this invention, the external fixation element 118 may be attached to a pulling force 214 such as a weight or pulley after the sleeve 110 is placed on the external fixation element 118, which is shown by FIG. 51.

Although a pharmacological sleeve 110 according to embodiments of this present invention has been described in considerable detail with reference to specific embodiments, the present invention is not confined to these embodiments. The invention extends to all variations and equivalents thereof within the scope of the foregoing description, the accompanying drawings, and the following claims.

What is claimed is:

1. A device for reducing potential complications from an elongated member inserted percutaneously into a bone structure through a tract, comprising:
a. a sleeve adapted to surround at least a predetermined length of the member, including in the vicinity of where the member is adapted to penetrate the skin;
b. a pharmacological agent, at least some of which is adapted to migrate into a portion of the tract; and
c. a spacer extending along part of the length of the sleeve, the spacer adapted to abut the sleeve and the member.

2. The device of claim 1, wherein the sleeve is a tubular structure having a circular cross-section.

3. The device of claim 1, wherein the sleeve is a hollow elongated structure having a non-circular cross-section.

4. The device of claim 1, wherein the predetermined length corresponds to at least a portion of the tract into which the member is inserted.

5. The device of claim 1, wherein the pharmacological agent is an antimicrobial agent.

6. The device of claim 1, wherein the pharmacological agent coats at least part of the outer surface of the sleeve.

7. The device of claim 1, wherein the pharmacological agent is suffused into at least part of the outer surface of the sleeve.

8. The device of claim 1, wherein the spacer and the sleeve form a single structure.

9. The device of claim 1, wherein the spacer and the sleeve do not form a single structure.

10. The device of claim 1, wherein the spacer comprises at least one spline.

11. The device of claim 1, wherein the spacer comprises a plurality of structures which in cross-section abut the sleeve and the elongated member in a substantially symmetrical fashion.

12. The device of claim 1, wherein the spacer has a semi-circular cross-section.

13. The device of claim 1, wherein the spacer has a non-semi-circular cross-section.

14. The device of claim 1, further comprising a positioning mechanism adapted to position the sleeve on the elongated member.

15. The device of claim 14, wherein the positioning mechanism comprises one of a heat shrink taper, a rolled edge, excess coating, an indentation, crimps, a pigtail, a heat shrinkable nose cone, a wedge, a retainer ring, heat shrink tubing, a hose clamp, a tapered connector, a cap, a cap with a tapered seal, a retainer band, a toothed hinge clamp, a cap with an O-ring, a clip with two interlocking arms, a snap-on clip, an O-ring, a clip with a V-shaped body, a C-clamp, a disk clip, a set screw clamp, a toothed clamp, a toothed clip, and a hinged clip.

16. A device for reducing potential complications from an elongated member inserted percutaneously into a bone structure through a tract, comprising:
a. a sleeve adapted to surround at least a predetermined length of the member, including in the vicinity of where the member is adapted to penetrate the skin;
b. a pharmacological agent, at least some of which is adapted to migrate into a portion of the tract; and
c. a positioning mechanism adapted to position the sleeve on the elongated member.

17. The device of claim 16, wherein the sleeve is a tubular structure having a circular cross-section.

18. The device of claim 16, wherein the sleeve is a hollow elongated structure having a non-circular cross-section.

19. The device of claim 16, wherein the predetermined length corresponds to at least a portion of the tract into which the member is inserted.

20. The device of claim 16, wherein the pharmacological agent is an antimicrobial agent.

21. The device of claim 16, wherein the pharmacological agent coats at least part of the outer surface of the sleeve.

22. The device of claim 16, wherein the pharmacological agent is suffused into at least part of the outer surface of the sleeve.

23. A device for releasing tissue from around an elongated member inserted percutaneously into a bone structure through a tract, comprising:
a. a cannulated elongated rod having indicia along an outer surface of the rod; and
b. a handle attached at an end of the rod, wherein the rod is adapted to receive the elongated member.

24. A system for reducing potential complications from an elongated member inserted percutaneously into a bone structure through a tract, comprising:
a. a sleeve surrounding at least a predetermined length of said elongated member, including in the vicinity of where the member is adapted to penetrate the skin, wherein said sleeve comprises:
(i). a pharmacological agent, at least some of which is adapted to migrate into a portion of the tract;
(ii). a spacer extending part of the length of the sleeve, the spacer adapted to abut the sleeve and the member;
b. said elongated member percutaneously inserted into said bone structure.

25. The system of claim 24, wherein the sleeve is a tubular structure having a circular cross-section.

26. The system of claim 24, wherein the sleeve is a hollow elongated structure, having a noncircular cross-section.

27. The system of claim 24, wherein the predetermined length corresponds to at least a portion of the tract into which the member is inserted.

28. The system of claim 24, wherein the pharmacological agent is an antimicrobial agent.

29. The system of claim 24, wherein the pharmacological agent coats at least part of the outer surface of the sleeve.

30. The system of claim 24, wherein the pharmacological agent is suffused into at least part of the outer surface of the sleeve.

31. The system of claim 24, wherein the spacer and the sleeve form a single structure.

32. The system of claim 24, wherein the spacer and the sleeve do not form a single structure.

33. The system of claim 24, wherein the spacer comprises at least one spline.

34. The system of claim 24, wherein the spacer comprises a plurality of structures which in cross-section abut the sleeve and the elongated member in a substantially symmetrical fashion.

35. The system of claim 24, wherein the spacer has a semi-circular cross-section.

36. The system of claim 24, wherein the spacer has a non-semi-circular cross-section.

37. The system of claim 24, wherein the sleeve further comprises a positioning mechanism adapted to position the sleeve on the elongated member.

38. The system of claim 24, wherein the positioning mechanism comprises on of a heat shrink taper, a rolled edge, excess coating, an indentation, crimps, a pigtail, a heat shrinkable nose cone, a wedge, a retainer ring, heat shrink tubing, a hose clamp, a tapered connector, a cap, a cap with a tapered seal, a retainer band, a toothed hinge clamp, a cap with an O-ring, a clip with two interlocking arms, a snap-on clip, an O-ring, a clip with a V-shaped body, a C-clamp, a disk clip, a set screw clamp, a toothed clamp, a toothed clip, and a hinged clip.

39. The system of claim 24, further comprising a device for releasing tissue from around the elongated member, comprising:
a. a cannulated elongated rod having indicia along an outer surface of the rod; and
b. a handle attached at an end of the rod,
wherein the rod is adapted to receive the elongated member.

40. A system for reducing potential complications from an elongated member inserted percutaneously into a bone structure through a tract, comprising:
a. a sleeve surrounding at least a predetermined length of said elongated member, including in the vicinity of where the member is adapted to penetrate the skin, wherein said sleeve comprises:
(i). a pharmacological agent, at least some of which is adapted to migrate into a portion of the tract;
(ii). a spacer extending part of the length of the sleeve, the spacer adapted to abut the sleeve and the member;
b. a bone fixator apparatus, wherein said elongated member is transversely attached to the bone fixator apparatus.

41. The system of claim 40, wherein the sleeve is a tubular structure having a circular cross-section.

42. The system of claim 40, wherein the sleeve is a hollow elongated structure having a non-circular cross-section.

43. The system of claim 40, wherein the predetermined length corresponds to at least a portion of the tract into which the member is inserted.

44. The system of claim 40, wherein the pharmacological agent is an antimicrobial agent.

45. The system of claim 40, wherein the pharmacological agent coats at least part of the outer surface of the sleeve.

46. The system of claim 40, wherein the pharmacological agent is suffused into at least part of the outer surface of the sleeve.

47. The system of claim 40, wherein the spacer and the sleeve form a single structure.

48. The system of claim 40, wherein the spacer and the sleeve do not form a single structure.

49. The system of claim 40, wherein the spacer comprises at least one spline.

50. The system of claim 40, wherein the spacer comprises a plurality of structures which in cross-section abut the sleeve and the elongated member in a substantially symmetrical fashion.

51. The system of claim 40, wherein the spacer has a semi-circular cross-section.

52. The system of claim 40, wherein the spacer has a non-semi-circular cross-section.

53. The system of claim 40, wherein the sleeve further comprises a positioning mechanism adapted to position the sleeve on the elongated member.

54. The system of claim 40, wherein the positioning mechanism comprises on of a heat shrink taper, a rolled edge, excess coating, an indentation, crimps, a pigtail, a heat shrinkable nose cone, a wedge, a retainer ring, heat shrink tubing, a hose clamp, a tapered connector, a cap, a cap with a tapered seal, a retainer band, a toothed hinge clamp, a cap with an O-ring, a clip with two interlocking arms, a snap-on clip, an O-ring, a clip with a V-shaped body, a C-clamp, a disk clip, a set screw clamp, a toothed clamp, a toothed clip, and a hinged clip.

55. The system of claim 40, further comprising a device for releasing tissue from around the elongated member, comprising:
a. a cannulated elongated rod having indicia along an outer surface of the rod; and
b. a handle attached at an end of the rod,
wherein the rod is adapted to receive the elongated member.

56. A process for reducing potential complications from an elongated member inserted percutaneously into a bone structure through a tract, comprising:
a. measuring the proper length of a sleeve, said sleeve adapted to surround at least a predetermined length of the member, including in the vicinity of where the member is adapted to penetrate the skin, said sleeve further comprising:
(i). a pharmacological agent, at least some of which is adapted to migrate into a portion of the tract;
(ii). a spacer extending part of the length of the sleeve, the spacer adapted to abut the sleeve and the member;
b. cutting the sleeve to the proper predetermined length;
c. placing the sleeve onto said elongated member; and
d. pushing the sleeve down the length of the elongated member, through subcutaneous tissue, to the vicinity of the bone.

57. The process of claim 57, wherein the sleeve is a tubular structure having a circular cross-section.

58. The process of claim 57, wherein the sleeve is a hollow elongated structure, having a non-circular cross-section.

59. The process of claim 57, wherein the predetermined length corresponds to at least a portion of the tract into which the member is inserted.

60. The process of claim 57, wherein the pharmacological agent is an antimicrobial agent.

61. The process of claim 57, wherein the pharmacological agent coats at least part of the outer surface of the sleeve.

62. The process of claim 57, wherein the pharmacological agent is suffused into at least part of the outer surface of the sleeve.

63. The process of claim 57, wherein the spacer and the sleeve form a single structure.

64. The process of claim 57, wherein the spacer and the sleeve do not form a single structure.

65. The process of claim 57, wherein the spacer comprises at least one spline.

66. The process of claim 57, wherein the spacer comprises a plurality of structures which in cross-section abut the sleeve and the elongated member in a substantially symmetrical fashion.

67. The process of claim 57, wherein the spacer has a semi-circular cross-section.

68. The process of claim 57, wherein the spacer has a non-semi-circular cross-section.

69. The process of claim 57, wherein the sleeve further comprises a positioning mechanism adapted to position the sleeve on the elongated member.

70. The process of claim 57, wherein the positioning mechanism comprises on of a heat shrink taper, a rolled edge, excess coating, an indentation, crimps, a pigtail, a heat shrinkable nose cone, a wedge, a retainer ring, heat shrink tubing, a hose clamp, a tapered connector, a cap, a cap with a tapered seal, a retainer band, a toothed hinge clamp, a cap with an O-ring, a clip with two interlocking arms, a snap-on clip, an O-ring, a clip with a V-shaped body, a C-clamp, a disk clip, a set screw clamp, a toothed clamp, a toothed clip, and a hinged clip.

71. The process of claim 57, wherein the measuring is performed with a device for releasing tissue from around the elongated member, comprising:
a. a cannulated elongated rod having indicia along an outer surface of the rod; and
b. a handle attached at an end of the rod,
wherein the rod is adapted to receive the elongated member.

72. A process for reducing potential complications from an elongated member inserted percutaneously into a bone structure through a tract, comprising:
a. measuring the proper length of a sleeve, said sleeve adapted to surround at least a predetermined length of the member, including in the vicinity of where the member is adapted to penetrate the skin, said sleeve further comprising:
(i). a pharmacological agent, at least some of which is adapted to migrate into a portion of the tract;
(ii). a spacer extending part of the length of the sleeve, the spacer adapted to abut the sleeve and the member;
b. cutting the sleeve to the proper predetermined length;
c. placing the sleeve onto said elongated member;
d. pushing the sleeve down the length of the elongated member, through subcutaneous tissue, to the bone;
e. tightening a fastening mechanism to apply tension to the elongated member; and
f. attaching the elongated member to a bone fixator apparatus.

73. The process of claim 72, wherein the sleeve is a tubular structure having a circular cross-section.

74. The process of claim 72, wherein the sleeve is a hollow elongated structure, having a non-circular cross-section.

75. The process of claim 72, wherein the predetermined length corresponds to at least a portion of the tract into which the member is inserted.

76. The process of claim 72, wherein the pharmacological agent is an antimicrobial agent.

77. The process of claim 72, wherein the pharmacological agent coats at least part of the outer surface of the sleeve.

78. The process of claim 72, wherein the pharmacological agent is suffused into at least part of the outer surface of the sleeve.

79. The process of claim 72, wherein the spacer and the sleeve form a single structure.

80. The process of claim 72, wherein the spacer and the sleeve do not form a single structure.

81. The process of claim 72, wherein the spacer comprises at least one spline.

82. The process of claim 72, wherein the spacer comprises a plurality of structures which in cross-section abut the sleeve and the elongated member in a substantially symmetrical fashion.

83. The process of claim 72, wherein the spacer has a semi-circular cross-section.

84. The process of claim 72, wherein the spacer has anon-semi-circular cross-section.

85. The process of claim 72, wherein the sleeve further comprises a positioning mechanism adapted to position the sleeve on the elongated member.

86. The process of claim 72, wherein the positioning mechanism comprises on of a heat shrink taper, a rolled edge, excess coating, an indentation, crimps, a pigtail, a heat shrinkable nose cone, a wedge, a retainer ring, heat shrink tubing, a hose clamp, a tapered connector, a cap, a cap with a tapered seal, a retainer band, a toothed hinge clamp, a cap with an O-ring, a clip with two interlocking arms, a snap-on clip, an O-ring, a clip with a V-shaped body, a C-clamp, a disk clip, a set screw clamp, a toothed clamp, a toothed clip, and a hinged clip.

87. The process of claim 72, wherein the measuring is performed with a device for releasing tissue from around the elongated member, comprising:
a. a cannulated elongated rod having indicia along an outer surface of the rod; and
b. a handle attached at an end of the rod,
wherein the rod is adapted to receive the elongated member.
